(19) United States
(12) Patent Application Publication
(10) Pub. No.: US 2024/0300886 A1
(43) Pub. Date: Sep. 12, 2024

(54) ARYLCYCLOHEXYLAMINE DERIVATIVES AND THEIR USE IN THE TREATMENT OF PSYCHIATRIC DISORDERS

(71) Applicant: GILGAMESH PHARMACEUTICALS, INC., New York, NY (US)

(72) Inventor: Andrew Carry KRUEGEL, New York, NY (US)

(73) Assignee: GILGAMESH PHARMACEUTICALS, INC., New York, NY (US)

(21) Appl. No.: 18/572,861

(22) PCT Filed: Jun. 27, 2022
(86) PCT No.: PCT/US2022/035179

§ 371 Date: Dec. 21, 2023

Related U.S. Application Data
(60) Provisional application No. 63/215,151, filed on Jun. 25, 2021.

(51) Int. Cl. C07C 225/20; A61K 31/137

(52) U.S. Cl. CPC C07C 225/20; A61K 31/137; C07B 2200/07

(57) ABSTRACT

Provided herein are arylcyclohexylamines and their use in the treatment of psychiatric disorders.

### BACKGROUND

Approximately one third of patients with major depressive disorder (MDD) fail to achieve remission of their symptoms, even after multiple rounds of treatment with several known classes of antidepressants, including selective serotonin reuptake inhibitors (SSRIs) (Rush et al. 2006). This high prevalence oftreatment-resistant depression (TRD) makes clear the need for new, more efficacious pharmacotherapies for depression that will target new mechanisms and/or patient populations. In recent years, ketamine, a drug long used as a dissociative anesthetic, has attracted considerable attention for its secondary use as a rapid-acting antidepressant with robust efficacy, even in patients with TRD (Zarate et al. 2006: Berman et al. 2000). The antidepressant effects of the drug are also notable in that they persist for days or weeks after a single administration. Importantly, the S enantiomer of ketamine (S-ket) has recently been approved by the United States Food and Drug Administration as a treatment for depression

Unfortunately, the potent dissociative anesthetic effects of ketamine and S-ket make these drugs attractive to recreational drug users and limit the broad clinical utility of these compounds by restricting their use to circumstances under the direct supervision of a medical provider. Given that the primary molecular target of ketamine is the N-methyl-D-aspartate receptor (NMDAR), inhibition of which is responsible for the drugs anesthetic effects, many have proposed that inhibition of this target is also responsible for the antidepressant effects of ketamine. Such a mechanism suggests that the antidepressant effects and dissociative effects of ketamine might be inseparable at the mechanistic level. However, a number of lines of evidence question this hypothesis (Aleksandrova et al. 2017). First, the R enantiomer of ketamine (R-ket), has been found to be more efficacious and longer lasting as an antidepressant in rodent models than S-ket, despite the fact that R-ket has a weaker binding affinity for NMDAR than S-ket (Zhang et al. 2014). Similarly, the ketamine metabolite (2R,6R)-hydroxynorketamine (HNK) has been shown to induce antidepressant effects in rodent models, but only weakly binds NMDAR and does not engage this receptor in vivo at dose levels that induce antidepressant effects (Zanos et al. 2016; Lumsden et al. 2019: Morris et al. 2017). Accordingly, both R-ket and HNK may induce antidepressant effects while limiting the dissociative effects of ketamine.

However, other strategies proposed to attenuate the dissociative effects of ketamine, for example, by targeting the NR2B subunit of NMDAR or utilizing a compound with low-trapping properties, have met with poor results. For example, a number of such structurally distinct NMDAR antagonists (e.g. memantine, MK-0657, and lanicemine), although in some cases reducing dissociation, have been found to be less efficacious and/or shorter acting than ketamine in treating depression (Zanos et al. 2016; Qu et al. 2017; Cerecor 2019; Kadriu et al. 2019; Lepow et al. 2017). Likewise, agonists with higher affinity for NMDAR (e.g. MK-801) or targeting alternative binding sites on the channel (e.g. rapastinel), have also met With failure (Yang et al. 2016; Al Idrus 2019). Accordingly, the precise molecular mechanisms underpinning the antidepressant effects of ketamine remain poorly understood and may involve other as-yet-unidentified targets. Further, the antidepressant effects of NMDAR modulators and the magnitude of their concomitant dissociative effects are in general highly unpredictable. At the same time, these findings have raised the exciting possibility that the antidepressant effects of ketamine might in fact be separable from its dissociative anesthetic effects.

In addition to its dissociative side effects, the use of ketanmine for depression treatment is further limited by the drug's poor oral bioavailability (Clements et al. 1982). Accordingly, for the treatment of MDD, ketamine is used almost entirely by the intravenous (i.v.) route. The practical challenges of i.v. administration further necessitate the use of ketamine under the supervision of a medical provider in a clinic or hospital setting. The inability to use ketamine by an oral route of administration is thus a major shortcoming that has limited the drug's broad adoption and increased medical costs associated with its use. Although other NMDAR antagonists have been developed that are orally bioavailable, to date none have reached the market, nor have they demonstrated the robust clinical efficacy of ketamine as an antidepressant. Therefore, there remains an acute need for novel antidepressants of the ketamine class that possess robust efficacy, decreased dissociative side effects, and increased oral bioavailability. A drug that retained the antidepressant activity of ketamine while also decreasing its dissociative effects and increasing oral bioavailability would provide a treatment option that was simpler to administer and potentially viable for at home use by virtue of its reduced dissociative effects and concomitant reduced abuse potential.

### SUMMARY OF THE PRESENT DISCLOSURE

The present disclosure, at least in part, provides arylcyclohexylamine compounds and compositions of single enantiomers or enantiomerically enriched mixtures of arylcyclohexylamines having significantly higher oral bioavailability, higher antidepressant potency, and/or greater therapeutic index between antidepressant effects and side effects, compared to ketamine.

For example, the disclosure provides for compounds having increased oral bioavailability, e.g., by having structural components that provide increased resistance to hepatic metabolism as compared to ketamine. This can be seen, for example, in their greater stability in both rodent and human liver microsome preparations. Importantly, despite such increases in oral bioavailability, disclosed compounds retain substantially short half-lives, in contrast to the more typical observation that increased hepatic stability may result in slow clearance. A short half-life may be desirable since therapeutic efficacy of such compounds may not depend on sustained receptor occupancy. Instead, pulsatile engagement of NMDAR (or other) signaling may be sufficient to induce therapeutic effects that last well beyond (days or weeks) the elimination of the drug (hours), thereby limiting overall exposure and reducing the duration of any dissociative or other negative side effects.

Further, in some embodiments, provided herein are compounds with increased antidepressant potency as a secondary effect of increased exposure, particularly after oral dosing and while retaining the high brain permeability of ketamine. Such compounds may be more potent as antidepressants even in cases where the in vitro affinity at NMDAR is similar to or lower than that of ketamine. Further, compounds provided herein may exhibit increased therapeutic index between antidepressant effects and dissociative side effects, as a consequence of NMDAR binding affinity of ˜1-5 μM, as determined though displacement of the radioligand [³H]MK-801 from NMDAR-containing membranes isolated from rat cortex. In certain embodiments, this affinity range may be useful in balancing the antidepressant efficacy and side effects, likely due to the rapid off kinetics of such compounds. For example, compounds with too high an affinity at NMDAR (<1 μM), for example racemic ketamine and S-ket, exhibit pronounced dissociative effects that restrict their use to physician-supervised settings and increase their abuse liability. Further, high affinity at NMDAR ray also decrease therapeutic efficacy in depression (e.g., both MK-801 and S-ket appear to exhibit weaker and less durable antidepressant effects than racemic ketamine and R-ket, which have lower affinities). In contrast, compounds with too low an affinity at NMDAR (>5 μM) may lose antidepressant efficacy, even when doses are appropriately scaled to account for such lower affinity. Further, even if efficacious, the very high doses required with such low potency compounds may exacerbate toxicological challenges or result in the introduction of undesirable off targets (as selectivity over other weak binding partners decreases).

Provided herein is a substantially enantiomerically pure compound selected from the group consisting of:

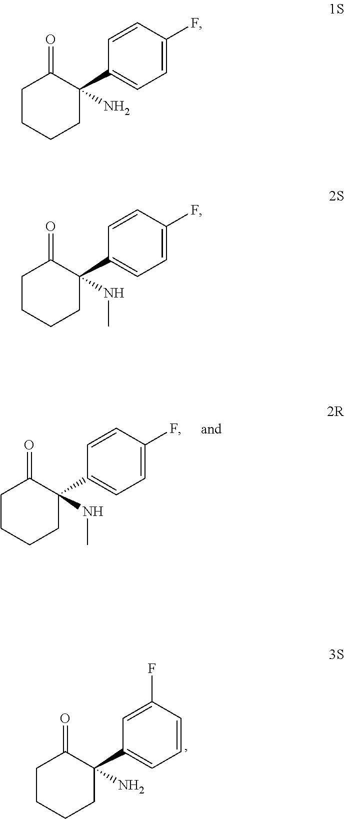 [SMILES: N[C@]1(c2ccc(F)cc2)CCCCC1=O ; CN[C@]1(c2ccc(F)cc2)CCCCC1=O ; CN[C@@]1(c2ccc(F)cc2)CCCCC1=O ; N[C@]1(c2cccc(F)c2)CCCCC1=O]

or a pharmaceutically acceptable salt thereof.

Also provided herein is an enantiomeric compound selected from the group consisting of

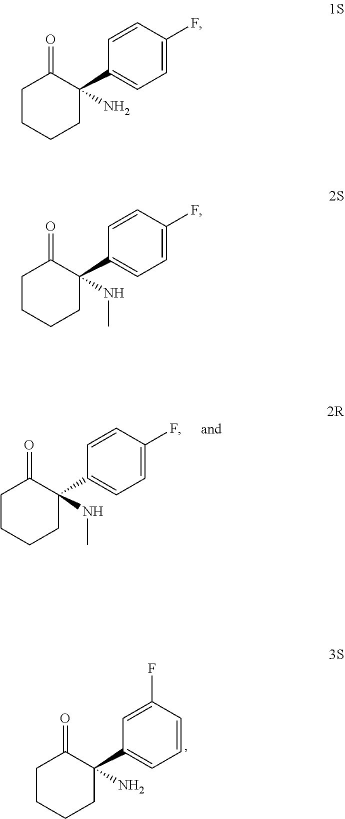 [SMILES: N[C@]1(c2ccc(F)cc2)CCCCC1=O ; CN[C@]1(c2ccc(F)cc2)CCCCC1=O ; CN[C@@]1(c2ccc(F)cc2)CCCCC1=O ; N[C@]1(c2cccc(F)c2)CCCCC1=O]

or a pharmaceutically acceptable salt thereof, wherein the enantiomeric compound is present in an enantiomeric mixture having at least 90%, at least 95%, or at least 99% of the enantiomeric compound.

Also provided herein is a composition comprising an enantiomeric mixture of a compound selected from the group consisting of:

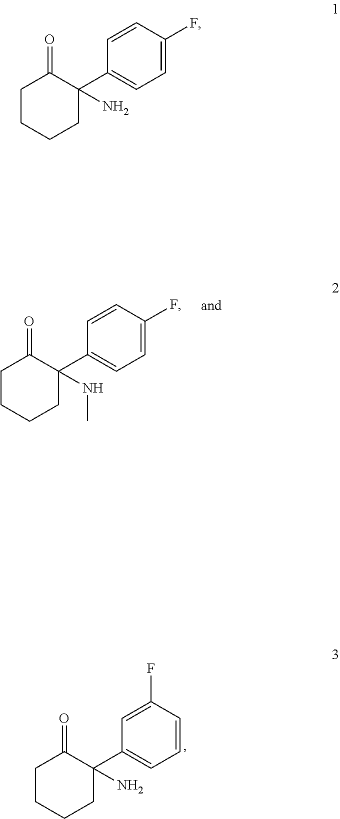 [SMILES: NC1(c2ccc(F)cc2)CCCCC1=O ; CNC1(c2ccc(F)cc2)CCCCC1=O ; NC1(c2cccc(F)c2)CCCCC1=O]

or a pharmaceutically acceptable salt thereof, wherein the enantiomeric mixture has a significantly greater amount of the enantiomer having the higher binding affinity at the NMDA receptor MK-801 site.

Also provided herein is a composition comprising an enantiomeric mixture of the compound

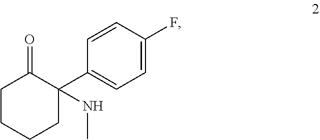 [SMILES: CNC1(c2ccc(F)cc2)CCCCC1=O]

or a pharmaceutically acceptable salt thereof, wherein the enantiomeric mixture has a significantly greater amount of the enantiomer having the lower binding affinity at the NMDA receptor MK-801 site.

Also provided herein is a method of treating depression, anxious depression, a mood disorder, an anxiety disorder, or a substance use disorder and any symptom or disorders associated therewith in a subject in need thereof the method comprising administering to the subject in need thereof an effective amount of a compound selected from the group consisting of:

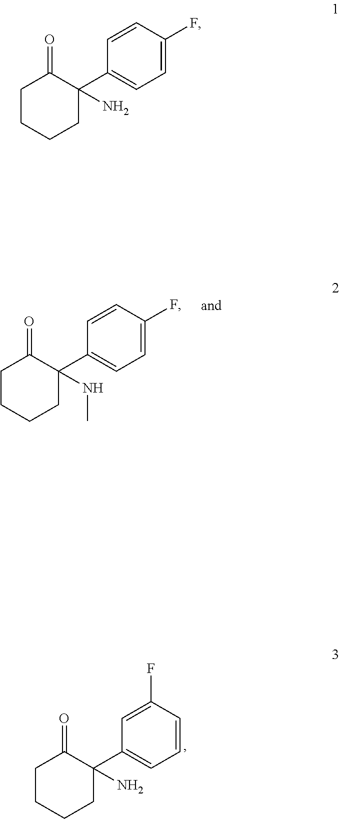 [SMILES: NC1(c2ccc(F)cc2)CCCCC1=O ; CNC1(c2ccc(F)cc2)CCCCC1=O ; NC1(c2cccc(F)c2)CCCCC1=O]

or a pharmaceutically acceptable salt thereof.

### BRIEF DESCRIPTION OF THE DRAWINGS

FIG. 1 shows a bar graph illustrating immobility time in the FST. A one-way ANOVA revealed a significant main effect of treatment (F(9,90)=8.953, P<0.0001) on the total time spent immobile in the FST. Dunnett's multiple comparisons test was used to test if a group was significantly different from vehicle. All treatments except for Compound 2R at 1 mg/kg were significantly different from vehicle. *P<0.05, **P<0.01, ***P<0.001, ****P<0.0001 vs. vehicle, Error bars represent the SEM.

FIG. 2 shows a graph illustrating plasma PK profile of 2R and 7R and their metabolite 1R in Sprague-Dawley rats after oral administration. Error bars represent the SEM.

FIG. 3 shows a graph illustrating brain PK profile of 2R and 7R and their metabolite 1R in Sprague-Dawley rats after oral administration. Error bars represent the SEM.

### DETAILED DESCRIPTION

In the following detailed description, numerous specific details are set forth in order to provide a thorough understanding of the present disclosure. However, it will be understood by those skilled in the art that the present disclosure may be practiced without these specific details. In other instances, well-known methods, procedures, and components have not been described in detail so as not to obscure the present disclosure.

Provided herein is a substantially enantiomerically pure compound selected from the group consisting of:

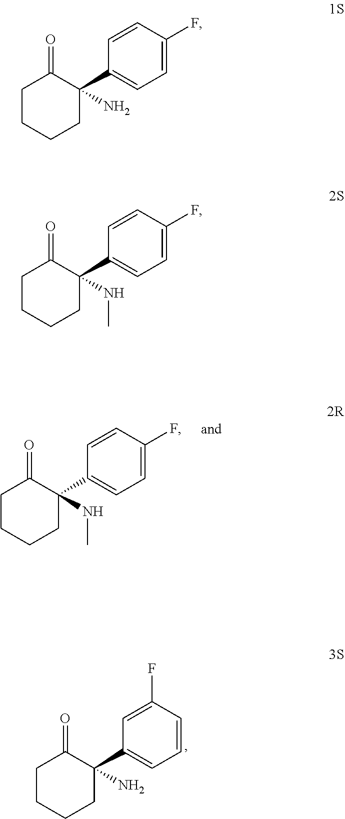 [SMILES: N[C@]1(c2ccc(F)cc2)CCCCC1=O ; CN[C@]1(c2ccc(F)cc2)CCCCC1=O ; CN[C@@]1(c2ccc(F)cc2)CCCCC1=O ; N[C@]1(c2cccc(F)c2)CCCCC1=O]

or a pharmaceutically acceptable salt thereof.

Also provided herein is an enantiomeric compound selected from the group consisting of:

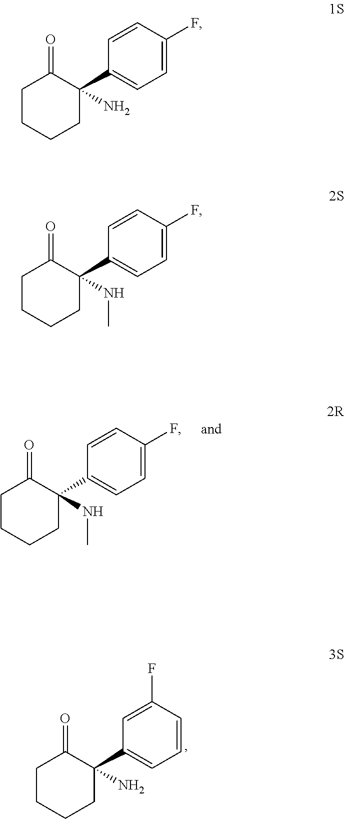 [SMILES: N[C@]1(c2ccc(F)cc2)CCCCC1=O ; CN[C@]1(c2ccc(F)cc2)CCCCC1=O ; CN[C@@]1(c2ccc(F)cc2)CCCCC1=O ; N[C@]1(c2cccc(F)c2)CCCCC1=O]

or a pharmaceutically acceptable salt thereof, wherein the enantiomeric compound is present in an enantiomeric mixture having at least 90%, at least 95%, or at least 99% of the enantiomeric compound.

In some embodiments, the compound is:

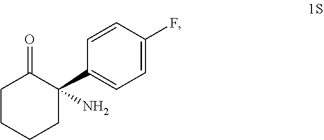 [SMILES: N[C@]1(c2ccc(F)cc2)CCCCC1=O]

or a pharmaceutically acceptable salt thereof.

In some embodiments, the compound is:

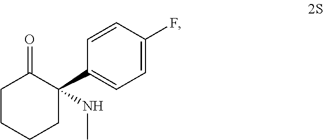 [SMILES: CN[C@]1(c2ccc(F)cc2)CCCCC1=O]

or a pharmaceutically acceptable salt thereof.

In some embodiments, the compound is:

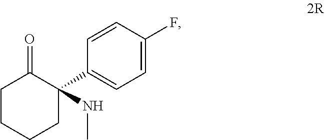 [SMILES: CN[C@@]1(c2ccc(F)cc2)CCCCC1=O]

or a pharmaceutically acceptable salt thereof.

In some embodiments, the compound is:

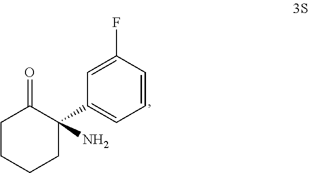 [SMILES: N[C@@]1(c2cccc(F)c2)CCCCC1=O]

or a pharmaceutically acceptable salt thereof.

In some embodiments, a pharmaceutical composition comprising a disclosed compound and a pharmaceutically acceptable excipient.

In some embodiments, the pharmaceutical composition is an oral composition.

Also provided herein is a composition comprising an enantiomeric mixture of a compound selected from the group consisting of:

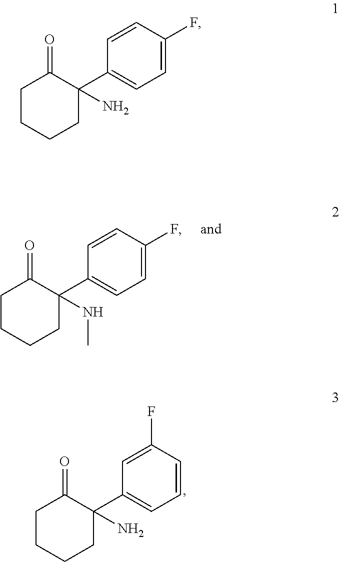 [SMILES: NC1(c2ccc(F)cc2)CCCCC1=O ; CNC1(c2ccc(F)cc2)CCCCC1=O ; NC1(c2cccc(F)c2)CCCCC1=O]

or a pharmaceutically acceptable salt thereof, wherein the enantiomeric mixture has a significantly greater amount of the enantiomer having the higher binding affinity at the NMDA receptor MK-801 site.

Also provided herein is a composition comprising an enantiomeric mixture of the compound:

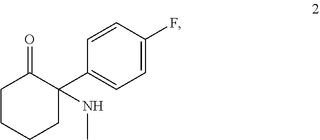 [SMILES: CNC1(c2ccc(F)cc2)CCCCC1=O]

or a pharmaceutically acceptable salt thereof, wherein the enantiomeric mixture has a significantly greater amount of the enantiomer having the lower binding affinity at the NMDA receptor MK-801 site.

In some embodiments, a method of treating depression, anxious depression, a mood disorder, an anxiety disorder, or a substance use disorder and any symptom or disorders associated therewith in a subject in need thereof, the method comprising administering to the subject in need thereof an effective amount of a disclosed compound or composition.

In some embodiments, the method of treatment wherein the compound or composition is orally administered.

In some embodiments, a method of treating depression or anxious depression in a subject in need thereof, the method comprising administering to the subject in need thereof an effective amount of a disclosed compound or composition.

In some embodiments, the method of treating depression or anxious depression wherein the compound or composition is orally administered.

Also provided herein is a method of treating depression, anxious depression, a mood disorder, an anxiety disorder, or a substance use disorder and any symptom or disorders associated therewith in a subject in need thereof the method comprising administering to the subject in need thereof an effective amount of a compound selected from the group consisting of:

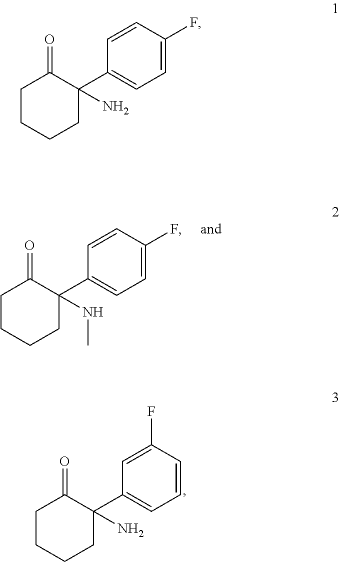 [SMILES: NC1(c2ccc(F)cc2)CCCCC1=O ; CNC1(c2ccc(F)cc2)CCCCC1=O ; NC1(c2cccc(F)c2)CCCCC1=O]

or a pharmaceutically acceptable salt thereof.

In some embodiments, the compound or composition is orally administered.

In some embodiments, the composition is a pharmaceutical composition.

Also provided herein is a compound selected from the group consisting of:

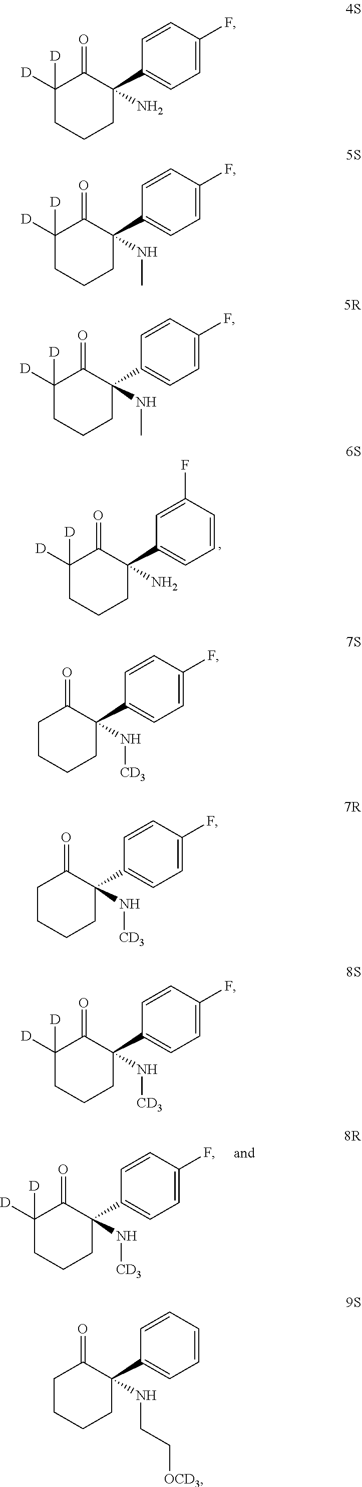

or a pharmaceutically acceptable salt thereof,
wherein D represents a deuterium-enriched H-site.

Also provided herein is a composition comprising a carrier and a compound selected from the group consisting of:

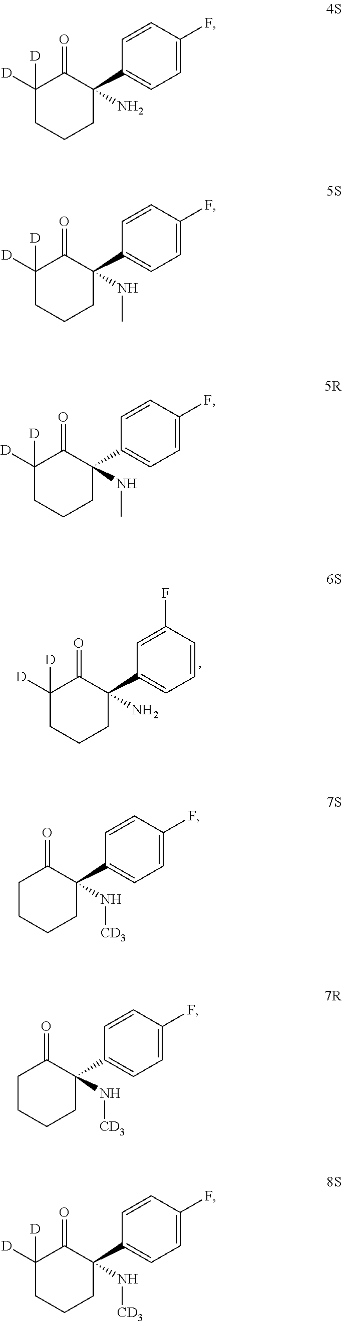

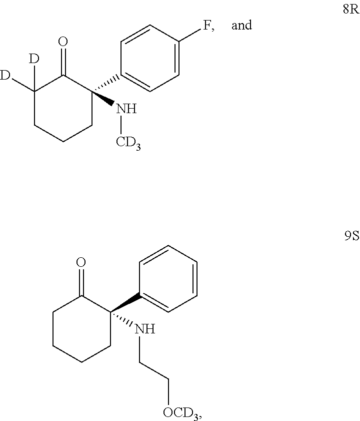

or a pharmaceutically acceptable salt thereof,
wherein D represents a deuterium-enriched H-site.

In some embodiments, each D represents a deuterium-enriched-H site and the level of deuterium at each deuterium-enriched-H site of the compound is 0.02% to 100%.

In some embodiments, each D represents a deuterium-enriched-H site and the level of deuterium at each deuterium-enriched-H site of the compound is 20%-100%, 50%-100%, 70%-100%, 90%-5100%, 95%-100%, 97%-100%, 98%-100%, or 99%-100%.

Also provided herein is a pharmaceutical composition comprising one or more compound disclosed herein and a pharmaceutically acceptable carrier.

In some embodiments, a composition described herein (e.g., a pharmaceutical composition) is an oral composition.

In some embodiments, the method wherein the composition is enriched in the compound over its opposite enantiomer.

In some embodiments, the optical purity of the compound is >5%, >25%, >50%, >75%, >90%, >95%, >97%, >98%, or >99%.

Also provided herein are compounds, methods, and compositions useful for treating refractory depression, e.g, patients suffering from a depressive disorder that does not, and/or has not, responded to adequate courses of at least one, or at least two, other antidepressant compounds or therapeutics. As used herein “depressive disorder” encompasses refractory depression.

In some embodiments, the compounds, methods, and compositions may be used to treat a psychiatric disorder including Bipolar and Related Disorders, e.g., Bipolar I Disorder, Bipolar II Disorder, Cyclothymic Disorder, Substance/Medication-Induced Bipolar and Related Disorder, and Bipolar and Related Disorder Due to Another Medical Condition.

In some embodiments, the compounds, methods, and compositions may be used to treat a psychiatric disorder including Substance-Related Disorders, e.g., preventing a substance use craving, diminishing a substance use craving, and/or facilitating substance use cessation or withdrawal. Substance use disorders involve abuse of psychoactive compounds such as alcohol, caffeine, cannabis, inhalants, opioids, sedatives, hypnotics, anxiolytics, stimulants, nicotine and tobacco. As used herein “substance” or “substances” are psychoactive compounds which can be addictive such as alcohol, caffeine, cannabis, hallucinogens, inhalants, opioids, sedatives, hypnotics, anxiolytics, stimulants, nicotine and tobacco. For example, the methods and compositions may be used to facilitate smoking cessation or cessation of opioid use.

In some embodiments, the compounds, methods, and compositions may be used to treat a psychiatric disorder including Anxiety Disorders, e.g., Separation Anxiety Disorder, Selective Mutism, Specific Phobia, Social Anxiety Disorder (Social Phobia), Panic Disorder, Panic Attack, Agoraphobia, Generalized Anxiety Disorder, Substance/Medication-Induced Anxiety Disorder, and Anxiety Disorder Due to Another Medical Condition.

In some embodiments, the compounds, methods, and compositions may be used to treat a psychiatric disorder including Obsessive-Compulsive and Related Disorders, e.g., Obsessive-Compulsive Disorder, Body Dysmorphic Disorder, Hoarding Disorder, Trichotillomania (Hair-Pulling Disorder), Excoriation (Skin-Picking) Disorder, Substance/Medication-Induced Obsessive-Compulsive and Related Disorder, and Obsessive-Compulsive and Related Disorder Due to Another Medical Condition.

In some embodiments, the compounds, methods, and compositions may be used to treat a psychiatric disorder including Trauma- and Stressor-Related Disorders, e.g., Reactive Attachment Disorder, Disinhibited Social Engagement Disorder, Posttraumatic Stress Disorder, Acute Stress Disorder, and Adjustment Disorders.

In some embodiments, the compounds, methods, and compositions may be used to treat a psychiatric disorder including Feeding and Eating Disorders, e.g., Anorexia Nervosa, Bulimia Nervosa, Binge-Eating Disorder, Pica, Rumination Disorder, and Avoidant/Restrictive Food Intake Disorder.

In some embodiments, the compounds, methods, and compositions may be used to treat a psychiatric disorder including Neurocognitive Disorders, e.g., Delirium, Major Neurocognitive Disorder, Mild Neurocognitive Disorder, Major or Mild Neurocognitive Disorder Due to Alzheimer's Disease, Major or Mild Frontotemporal Neurocognitive Disorder, Major or Mild Neurocognitive Disorder With Lewy Bodies, Major or Mild Vascular Neurocognitive Disorder, Major or Mild Neurocognitive Disorder Due to Traumatic Brain Injury, Substance/Medication-Induced Major or Mild Neurocognitive Disorder, Major or Mild Neurocognitive Disorder Due to HIV Infection, Major or Mild Neurocognitive Disorder Due to Prion Disease, Major or Mild Neurocognitive Disorder Due to Parkinson's Disease, Major or Mild Neurocognitive Disorder Due to Huntington's Disease, Major or Mild Neurocognitive Disorder Due to Another Medical Condition, and Major or Mild Neurocognitive Disorder Due to Multiple Etiologies.

In some embodiments, the compounds, methods, and compositions may be used to treat a psychiatric disorder including Neurodevelopmental Disorders, e.g., Autism Spectrum Disorder, Attention-Deficit/Hyperactivity Disorder, Stereotypic Movement Disorder, Tic Disorders, Tourette's Disorder, Persistent (Chronic) Motor or Vocal Tic Disorder, and Provisional Tic Disorder.

In some embodiments, the compounds, methods, and compositions may be used to treat a psychiatric disorder including Personality Disorders, e.g., Borderline Personality Disorder.

In some embodiments, the compounds, methods, and compositions may be used to treat a psychiatric disorder including Sexual Dysfunctions, e.g. Delayed Ejaculation, Erectile Disorder, Female Orgasmic Disorder, Female Sexual Interest/Arousal Disorder, Genito-Pelvic Pain/Penetration Disorder, Male Hypoactive Sexual Desire Disorder, Premature (Early) Ejaculation, and Substance/Medication-Induced Sexual Dysfunction.

In some embodiments, the compounds, methods, and compositions may be used to treat a psychiatric disorder including Gender Dysphoria, e.g, Gender Dysphoria.

The terms “effective amount” or “therapeutically effective amount” refer to an amount of a compound, material, composition, medicament, or other material that is effective to achieve a particular pharmacological and/or physiologic effect including but not limited to reducing the frequency or severity of sadness or lethargy, depressed mood, anxious or sad feelings, diminished interest in all or nearly all activities, significant increased or decreased appetite leading to weight gain or weight loss, insomnia, irritability fatigue, feelings of worthlessness, feelings of helplessness, inability to concentrate, and recurrent thoughts of death or suicide, or to provide a desired pharmacologic and/or physiologic effect, for example, reducing, inhibiting, or reversing one or more of the underlying pathophysiological mechanisms underlying the neurological dysfunction, modulating dopamine levels or signaling, modulating serotonin levels or signaling, modulating norepinephrine levels or signaling, modulating glutamate or GABA levels or signaling, modulating synaptic connectivity or neurogenesis in certain brain regions, or a combination thereof.

The term “therapeutic index” used in reference to any compound and its associated therapeutic effects and side effects refers to the ratio of the dose of said compound required to induce a particular negative side effect to the dose of said compound required to induce the desired therapeutic effect. For example, in the case of racemic ketamine, antidepressant therapeutic effects and dissociative side effects occur at similar doses and thus, the therapeutic index of this compound in this context is ˜1:1. In contrast, a compound disclosed herein might have an improved therapeutic index, for example 3:1, where a 3-fold higher dose is required to induce dissociative side effects relative to that needed for antidepressant therapeutic effects.

In some embodiments, methods include treating a psychiatric disorder by administering to a subject in need thereof a pharmaceutical composition including about 0.01 mg to about 400 mg of a compound disclosed herein. In some embodiments, doses may be, e.g., in the range of about 0.1 to 300 mg, 0.1 to 250 mg, 0.1 to 200 mg, 0.1 to 150 mg, 0.1 to 100 mg, 0.1 to 75 mg, 0.1 to 50 mg, 0.1 to 25 mg, 0.1 to 20 mg, 0.1 to 15 mg, 0.1 to 10 mg, 0.1 to 5 mg, 0.1 to 1 mg, 10 to 300 mg, 10 to 250 mg, 10 to 200 mg, 10 to 150 mg, 10 to 100 mg, 10 to 50 mg, 10 to 25 mg, 10 to 15 mg,, 20 to 300 mg, 20 to 250 mg, 20 to 200 mg, 20 to 150 mg, 20 to 100 mg, 20 to 50 mg, 50 to 300 mg, 50 to 250 mg, 50 to 200 mg, 50 to 150 mg, 50 to 100 mg, 100 to 300 mg 100 to 250 mg, 100 to 200 mg, with doses of, e.g., about 0.25 mg, 0.5 mg. 0.75 mg, 1 mg, 1.25 mg, 1.5 mg, 1.75 mg, 2.0 mg, 2.5 mg, 3.0 mg, 3.5 mg, 4.0 mg, 4.5 mg, 5 mg, 10 mg, 15 mg, 20 mg, 25 mg, 30 mg, 35 mg, 40 mg, 45 mg, 50 mg, 75 mg, 100 mg, 125 mg, 150 mg, 175 mg, 200 mg, 225 mg, 250 mg, 275 mg, 300 mg, and 400 mg being examples.

In some embodiments, dosages may include amounts of a compound disclosed herein or a pharmaceutically acceptable salt thereof in the range of about, e.g., 1 mg to 200 mg, 1 mg to 100 mg, 1 mg to 50 mg, 1 mg to 40 mg, 1 mg to 30 mg, 1 mg to 20 mg, 1 mg to 15 mg, 0.01 mg to 10 mg, 0.1 mg to 15 mg, 0.15 mg to 12.5 mg, or 0.2 mg to 10 mg, with doses of 0.1 mg, 0.2 mg, 0.3 mg, 0.4 mg, 0.5 mg, 0.6 mg, 0.7 mg, 0.8 mg, 0.9 mg, 1.5 mg, 1.0 mg, 1.75 mg, 2 mg, 2.5 mg, 2.75 mg, 3 mg, 3.5 mg, 3.75 mg, 4 mg, 4.5 mg, 4.75 mg, 5 mg, 5.5 mg, 6 mg, 6.5 mg, 7 mg, 7.5 mg, 8 mg, 8.5 mg, 9 mg, 10 mg, 11 mg, 12 mg, 15 mg, 20 mg, 25 mg, 30 mg, 35 mg, 40 mg, 45 mg, 50 mg, 60 mg, 75 mg, 80 mg, 90 mg, 100 mg, 125 mg, 150 mg, and 200 mg being specific examples of doses.

Typically, dosages of a compound disclosed herein or a pharmaceutically acceptable salt thereof, are administered once, twice, three or four times daily, every other day, every three days, once weekly, or once a month to a patient in need thereof. In some embodiments, the dosage is about, e.g., 1-400 mg/day, or 1-300 mg/day, or 1-250 mg/day, or 1-200 mg/day, for example 300 mg/day, 250 mg/day, 200 mg/day, 150 mg/day, 100 mg/day, 75 mg/day, 50 mg/day, 25 mg/day, 20 mg/day, 10 mg/day, 5 mg/day, or 1 mg/day.

In some embodiments, pharmaceutical compositions for parenteral or inhalation, e.g., a spray or mist of a compound of the present disclosure or a pharmaceutically acceptable salt thereof, include a concentration of about 0.005 mg/mL to about 500 mg/mL. In some embodiments, the compositions include a compound disclosed herein or a pharmaceutically acceptable salt thereof, at a concentration of, e.g., about 0.05 mg/mL to about 50 mg/mL, about 0.05 mg/mL to about 100 mg/mL, about 0.005 mg/mL to about 500 mg/mL, about 0.1 mg/mL to about 50 mg/ML, about 0.1 mg/mL to about 10 mg/mL, about 0.05 mg/mL to about 25 mg/mL, about 0.05 mg/mL to about 10 mg/mL, about 0.05 mg/mL to about 5 mg/mL, or about 0.05 mg/mL to about 1 mg/mL.

In some embodiments, the composition includes a compound disclosed herein or a pharmaceutically acceptable salt thereof, at a concentration of, e.g., about 0.05 mg/mL to about 15 mg/mL, about 0.5 mg/mL to about 10 mg/mL, about 0.25 mg/mL to about 5 mg/mL, about 0.5 mg/mL to about 7 mg/mL, about 1 mg/ML to about 10 mg/mL, about 5 mg/mL to about 10 mg/mL, about 5 mg/mL to about 15 mg/mL, about 5 mg/mL to 25 mg/mL, about 5 mg/mL to 50 mg/mL, or about 10 mg/mL to 100 mg/mL. In some embodiments, the pharmaceutical compositions are formulated as a total volume of about, e.g., 10 mL, 20 mL, 25 mL, 50 mL, 100 mL, 200 mL, 250 mL, or 500 mL.

Typically, dosages may be administered to a subject once, twice, three or four times daily, every other day, every three days, twice weekly, once weekly, twice monthly, or once monthly. In some embodiments, a compound disclosed herein is administered to a subject once in the morning, or once in the evening. In some embodiments, a compound disclosed herein is administered to a subject once in the morning, and once in the evening. In some embodiments, a disclosed herein is administered to a subject three tines a day (e.g., at breakfast, lunch, and dinner), at a dose, e.g., of 50 mg/administration (e.g., 150 mg/day).

In some embodiments, a compound disclosed herein is administered to a subject at a dose of 25 mg/day in one or more doses. In some embodiments, a compound disclosed herein is administered to a subject at a dose of 50 mg/day in one or more doses. In some embodiments, a compound disclosed herein is administered to a subject at a dose of 75 mg/day in one or more doses. In some embodiments, a compound disclosed herein is administered to a subject at a dose of 100 mg/day in one or more doses. In some embodiments, a compound disclosed herein is administered to a subject at a dose of 150 mg/day in one or more doses. In some embodiments, a compound disclosed herein is administered to a subject at a dose of 200 mg/day in one or more doses. In some embodiments, a compound disclosed herein is administered to a subject at a dose of 250 mg/day in one or more doses.

In some embodiments, the dosage of a compound disclosed herein is 0.01-100 mg/kg, 0.5-50 mg/kg, 0.5-10 mg/kg or 25-50 mg/kg once, twice, three times or four times daily. For example, in some embodiments, the dosage is 0.1 mg/kg, 0.25 mg/kg, 0.5 mg/kg, 1 mg/kg, 5 mg/kg, 7.5 mg/kg, or 10 mg/kg once, twice, three times or four times daily. In some embodiments, a subject is administered a total daily dose of 0.01 mg to 500 mg of a compound disclosed herein once, twice, three times, or four times daily. In some embodiments, the total amount administered to a subject in 24-hour period is, e.g., 5 mg, 10 mg, 20 mg, 25 mg, 30 mg, 35 mg, 40 mg, 45 mg, 50 mg, 60 mg, 75 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg. In some embodiments, the subject may be started at a low dose and the dosage is escalated. In some embodiments, the subject may be started at a high dose and the dosage is decreased.

In some embodiments, a compound or composition disclosed herein is administered to a patient under the supervision of a healthcare provider.

In some embodiments, a compound or composition disclosed herein is administered to a patient under the supervision of a healthcare provider at a clinic specializing in the delivery of psychoactive treatments.

In some embodiments, a compound or composition disclosed herein is administered to a patient under the supervision of a healthcare provider at a dose intended to induce a psychedelic experience in the subject.

In some embodiments, the administration to a patient under the supervision of a healthcare provider occurs periodically in order to maintain a therapeutic effect in the patient, e.g., every three days, twice weekly, once weekly, twice monthly, once monthly, thrice yearly, twice yearly, or once yearly.

In some embodiments, a compound or composition disclosed herein is administered by a patient on their own at home or otherwise away from the supervision of a healthcare provider.

In some embodiments, the administration by a patient on their own occurs periodically in order to maintain a therapeutic effect in the patient, e.g., daily, every other day, every three days, twice weekly, once weekly, twice monthly, or once monthly,

In some embodiments, a compound or composition disclosed herein may be administered at specified intervals. For example, during treatment a patient may be administered a compound or composition at intervals of every, e.g., 1 year, 6 months, 90 days. 60 days, 30 days, 14 days, 7 days, 3 days, 24 hours, 12 hours, 8 hours, 6 hours, 5 hours, 4 hours, 3 hours, 2.5 hours, 2.25 hours, 2 hours, 1.75 hours, 1.5 hours, 1.25 hours, 1 hour, 0.75 hour, 0.5 hour, or 0.25 hour.

In some embodiments, a compound disclosed herein is in the form of a pharmaceutically acceptable salt thereof.

In some embodiments, a pharmaceutical composition comprises one or more of the compounds disclosed herein.

In some embodiments, a salt of the compound disclosed herein is used in any of the methods, uses, or compositions.

In some embodiments, a pharmaceutically acceptable salt of the compound disclosed herein is used in any of the methods, uses, or compositions.

In some embodiments, an ester of the compound disclosed herein is used in any of the methods, uses, or compositions.

Any of the compounds disclosed herein may be used in any of the disclosed methods, uses, or compositions.

Any of the compounds used in the disclosed methods, uses, or compositions may be replaced with any other compound disclosed herein.

Any of the disclosed generic compounds may be used in any of the disclosed methods, uses, or compositions.

The terms “about” or “approximately” as used herein mean within an acceptable error range for the particular value as determined by one of ordinary skill in the art, which will depend in part on how the value is measured or determined, i.e., the limitations of the measurement system. For example, “about” can mean Within 3 or more than 3 standard deviations, per the practice in the art. Alternatively, “about” can mean a range of up to 20%, a range up to 10%, a range up to 5%, and/or a range up to 1% of a given value. Alternatively, particularly with respect to biological systems or processes, the term can mean within an order of magnitude, e.g., within 5-fold, or within 2-fold, of a value. “About” and “approximately” are used interchangeably herein.

Compounds disclosed herein may include at least one asymmetric center. These centers are designated by the symbols “R” or “S,” depending on the configuration of substituents around the chiral atom. Unless otherwise indicated in the structural formula, it should be understood that the present disclosure encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure contemplates all cis, trans, syn, anti, entgegen (E), and zusammen (Z) isomers as well as the appropriate mixtures thereof. Additionally, compounds may exist as tautomers; all tautomeric isomers are provided by the present disclosure. Additionally, the compounds disclosed herein can exist in unsolvated as well as solvated forms with pharmaceutically acceptable solvents such as water, ethanol, and the like. In general, the solvated forms are considered equivalent to the unsolvated forms.

In some embodiments, a composition disclosed herein may be enriched in a specific enantiomer of any compound disclosed herein relative to the corresponding opposite enantiomer of that compound, such that the mixture is not racemic. In such cases, the subject mixture of isomers is understood to have an enantiomeric excess and optical purity >0%. The enantiomeric excess or optical purity of the isomeric mixture may be >0%, >5%, >25%, >50%, >75%, >90%, >95%, >97%, >98%, or >99% The enantiomeric excess or optical purity of the isomeric mixture may 5-100%, 25-100%, 50-100%, 75-100%, 90-100%, 95-100%, 97-100%, 98-100%, or 99-100%. Thus, for example, contemplated herein is a composition including the S enantiomer of a compound substantially free of the R enantiomer, or the R enantiomer substantially free of the S enantiomer. Further, if the named compound includes more than one chiral center, the scope of the present disclosure also includes compositions including mixtures of varying proportions between the diastereomers, as well as compositions including one or more diastereomers substantially free of one or more of the other diastereomers. By “substantially free” it is meant that the composition includes less than 50%, 25%, 15%, 10%, 8%, 5%, 3%, 2%, or 1% of the minor enantiomer or diastereomer(s).

For clarity, in the context of the present disclosure, chemical structures of a compound depicted with a specific stereochemical orientation at any particular chiral center, as defined by wedge and dash notation, are intended to represent the specified stereoisomer of said compound in substantially pure form, or a mixture enriched in the stereoisomer(s) with the specified stereochemical orientation at the defined chiral center over the stereoisomer(s) with the opposite orientation at said chiral center.

The disclosure may also include any salt of a compound disclosed herein above and below, including any pharmaceutically acceptable salt, wherein a compound disclosed herein has a net charge (either positive or negative) and at least one counter ion (having a counter negative or positive charge) is added thereto to form said salt. The phrase “pharmaceutically acceptable salt(s)”, as used herein, means those salts of compounds disclosed herein that are safe and effective for pharmaceutical use in mammals and that possess the desired biological activity. Pharmaceutically acceptable salts include salts of acidic or basic groups present in compounds disclosed herein. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds disclosed herein can form pharmaceutically acceptable salts with various amino acids. Suitable base salts include, but are not limited to, aluminum, calcium, lithium, magnesium, potassium, sodium, zinc, and diethanolamnine salts. For a review on pharmaceutically acceptable salts see BERGE ET AL., 66 J. PHARM. SCI. 1-19 (1977), incorporated herein by reference.

The present disclosure is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include ¹³C and ¹⁴C.

It will be noted that any notation of a carbon in structures throughout this application, when used without further notation, are intended to represent all isotopes of carbon, such as ¹²C, ¹³C or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In some embodiments, each D in a chemical structure represents a deuterium-enriched-H site and the level of deuterium at each deuterium-enriched-H site of the compound is 0.02% to 100%.

In some embodiments, each D in a chemical structure represents a deuterium-enriched-H site and the level of deuterium at each deuterium-enriched-H site of the compound is 20-100%, 50-100%, 70-100%, 90-100%, 95-100%, 97-100%, or 99-100%.

It is understood that substituents and substitution patterns on the compounds used in the method of the present disclosure can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present disclosure, one of ordinary skill in the art will recognize that the various substituents, i.e. R₁, R₂, etc., are to be chosen in conformity with well-known principles of chemical structure connectivity.

The term “treatment” as used herein means the management and care of a patient for the purpose of combating a disease, disorder or condition. The term is intended to include the delaying of the progression of the disease, disorder or condition, the alleviation or relief of symptoms and complications, and/or the cure or elimination of the disease, disorder or condition. The patient to be treated is preferably a mammal, in particular a human being.

The present disclosure thus also relates to pharmaceutical compositions comprising a compound as defined herein below and above in admixture with pharmaceutically acceptable auxiliaries, and optionally other therapeutic agents. The auxiliaries must be “acceptable” in the sense of being compatible with the other ingredients of the composition and not deleterious to the recipients thereof.

Pharmaceutical compositions include those suitable for oral, rectal, nasal, topical (including transdermal, buccal and sublingual), vaginal or parenteral (including subcutaneous, intramuscular, intravenous and intradermal) administration or administration via an implant. The compositions may be prepared by any method well known in the art of pharmacy.

Such methods include the step of bringing in association compounds used in the present disclosure or combinations thereof with any auxiliary agent. The auxiliary agent(s), also named accessory ingredient(s), include those conventional in the art, such as carriers, fillers, binders, diluents, disintegrants, lubricants, colorants, flavoring agents, anti-oxidants, and wetting agents. Such auxiliary agents are suitably selected with respect to the intended form and route of administration and as consistent with conventional pharmaceutical practices.

Pharmaceutical compositions suitable for oral administration may be presented as discrete dosage units such as pills, tablets, dragées or capsules, or as a powder or granules, or as a solution or suspension. The active ingredient may also be presented as a bolus or paste. The compositions can further be processed into a suppository or enema for rectal administration.

Tablets may contain the active ingredient compounds and suitable binders, lubricants, disintegrating agents, coloring agents, flavoring agents, flow-inducing agents, and melting agents. Gelatin capsules may contain the active ingredient compounds and powdered carriers, such as to lactose, starch, cellulose derivatives, magnesium stearate, stearic acid, and the like. Similar diluents can be used to make compressed tablets. Compressed tablets can be sugar coated or film coated to mask any unpleasant taste and protect the tablet from the atmosphere, or enteric coated for selective disintegration in the gastrointestinal tract. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Liquid dosage forms for oral administration can contain coloring and flavoring to increase patient acceptance.

For parenteral administration, suitable compositions include aqueous and non-aqueous sterile solutions. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA, In addition, parenteral solutions can contain preservatives, such as benzalkonium chloride, methyl- or propyl-paraben, and chlorobutanol. The compositions may be presented in unit-dose or multi-dose containers, for example sealed vials and ampoules, and may be stored in a freeze-dried (lyophilized) condition requiring only the addition of sterile liquid carrier, for example water, prior to use. For transdermal administration, e.g. gels, patches or sprays can be contemplated. Compositions or formulations suitable for pulmonary administration e.g. by nasal inhalation, include fine dusts or mists which may be generated by means of metered dose pressurized aerosols, nebulizers or insufflators. Parenteral and intravenous forms may also include minerals and other materials to make them compatible with the type of injection or delivery system chosen.

The compounds used in the method of the present disclosure may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present disclosure may also be coupled to soluble polymers as targetable drug carriers or as prodrugs. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Pharmaceutical compositions herein may be provided with immediate release, delayed release, extended release, or modified release profiles. In some embodiments, pharmaceutical compositions with different drug release profiles may be combined to create a two-phase or three-phase release profile. For example, pharmaceutical compositions may be provided with an immediate release and an extended release profile. In some embodiments, pharmaceutical compositions may be provided with an extended release and delayed release profile. Such composition may be provided as pulsatile formulations, multilayer tablets, or capsules containing tablets, beads, granules, etc.

Pharmaceutical compositions herein may be provided with abuse deterrent features by techniques know in the art, for example, by making a tablet that is difficult to crush or to dissolve in water.

The present disclosure further includes a pharmaceutical composition, as hereinbefore described, in combination with packaging material, including instructions for the use of the composition for a use as hereinbefore described.

The exact dose and regimen of administration of the composition will necessarily be dependent upon the type and magnitude of the therapeutic or nutritional effect to be achieved and may vary depending on factors such as the particular compound, formula, route of administration, or age and condition of the individual subject to whom the composition is to be administered.

Furthermore, in some embodiments a pharmaceutical composition disclosed herein may include a single enantiomer, diastereomer or structural isomer of a compound disclosed herein. In other embodiments, a pharmaceutical composition disclosed herein may include a mixture of at least one single enantiomer, diastereomer or structural isomer of a compound disclosed herein together with any other enantiomer, diastereomer or structural isomer of a compound disclosed herein. In further embodiments, said mixture is a racemic mixture. In other embodiments, said mixture is a non-racemic mixture (wherein one enantiomer or diastereomer is enriched in said non-racemic mixture).

The compounds used in the method of the present disclosure may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the sane route or by two or more different routes of administration depending on the dosage forms employed.

Each embodiment disclosed herein is contemplated as being applicable to each of the other disclosed embodiments. Thus, all combinations of the various elements described herein are within the scope of the disclosure.

It can be appreciated that stereochemical designations (e.g., R- and S-configurations for certain provided compounds below) may differ upon determination by e.g., X-ray crystallography.

### Example 1: Preparation of Compounds 1 and 2 and their Enantiomners

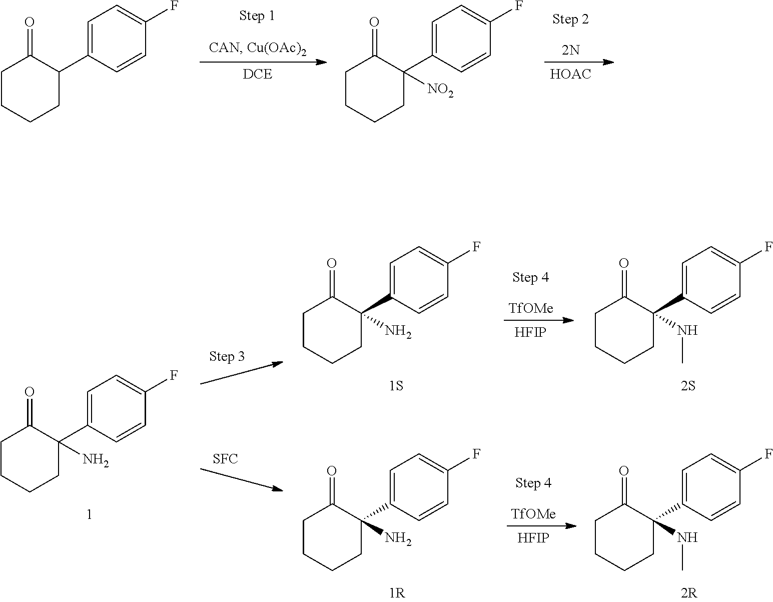

### Step 1: Preparation of 2-(4-fluorophenyl)-2-nitrocyclohexan-1-one

A mixture of 2-(4-fluorophenyl)cyclohexan-1-one (14 g 72.83 mmol, 1 eq). CAN (79.85 g, 145.66 mmol, 72.59 mL, 2 eq), and Cu(OAc)₂ (2.65 g, 14.57 mmol, 0.2 eq) in DCE (140 mL) was stirred at 85° C. for 12 h. On completion, the mixture was filtered and concentrated. The residue was purified by column chromatography (SiO₂, Petroleum ether/Ethyl acetate=100/1 to 0/1) to afford 2-(4-fluorophenyl)-2-nitrocyclohexan-1-one (6.1 g, 25.71 mmol, 35.31% yield) as a yellow solid. ¹H NMR (400 MHz, CHLOROFORM-d) δ=7.41-7.31 (m, 2H), 7.16 (t, J=8.4 Hz, 2H), 3.11 (ddd, J=3.6, 10.4, 14.0 Hz, 1H), 2.87-2.76 (m, 1H), 2.73-2.64 (m, 1H), 2.60-2.48 (m, 1H), 2.02-1.88 (m, 3H), 1.84-1.72 (m, 1H).

### Step 2: Preparation of 2-amino-2-4-fluorophenyl)cyclohexan-1-one (1)

To a mixture of 2-(4-fluorophenyl)-2-nitrocyclohexan-1-one (5.6 g, 23.61 mmol, 1 eq) in AcOH (10 mL) was added Zn (15.44 g, 236.06 mmol, 10 eq) in several portions and the resulting mixture was stirred at 30° C. for 12 h. On completion, the mixture was filtered and concentrated. The residue was dissolved in DCM (20 mL), washed with sat. aq. NaHCO₃ (10 mL), H₂O (5 mL), and brine (10 mL), dried over Na₂SO₄, filtered, and concentrated. The residue was purified by prep-HPLC (column: Agela DuraShell C18 (250 mm*80 mm, 10 μm); mobile phase: A: water (NH₄HCO₃), B: ACN; B %: 35%, 20 min) to afford 2-amino-2-(4-fluorophenyl)cyclohexan-1-one (2.9 g, 13.99 mmol, 59.28% yield, 1) as a brown oil. ¹H NMR (400 MHz, CHLOROFORM-d) δ=7.52-7.40 (m, 2H), 7.32 (br s, 1H), 7.34-7.20 (m, 21H), 2.93-2.92 (m, 1H), 3.08-2.92 (m, 1H), 2.74-2.63 (m, 1H), 2.63-2.50 (m, 1H), 2.28-2.16 (m, 1H), 2.10 (br s, 2H), 2.04-1.85 (m, 4H).

Note: The free base of this compound is unstable and dimerizes over time. It should be stored frozen or quickly converted to the HCl salt to prevent this.

### Step 3: Preparation of (S)-2-amino-2-(4-fluorophenyl)cyclohexan-1-one (1S) and (R)-2-amino-2-(4-fluorophenyl)cyclohexan-1-one (1R)

The racemate 1 (2.9 g) was as separated by SFC (column: DAICEL CHIRALPAK AD (250 mm*30 mm, 10 μm); mobile phase: A: CO₂, B: 0.1% NH₃H₂O in ETOH; B %. 27%, multi-injection process with 6-min spacing between injections) to afford ENT-1 free base (RT=2.266 min, 1.1 g, 1.62 mmol, 1S_FB) as a yellow oil and ENT-2 free base (RT=2.945 min, 1.1 g, 1.28 mmol, 1R_FB) as a yellow oil.

A portion of each free base was further purified by prep-HPLC (column: Welch Xtimate C18 (100 mm*25 mm, 3 μm); mobile phase: A: water (0.04% HC), B: ACN; B %: 1%-20%, 8 min) to afford ENT-1 HCl (RT=2.266 min, 272 mg, HCl salt, 1S) as a white solid and ENT-2 HCl (RT=2,945 min, 283 mg, HCl salt, 1R) as a white solid.

ENT-1 HCl, RT=2.266 min (assigned here as the S isomer, 1S); LCMS (R_{T}=1.449 min, MS calc.: 207.1, [M+H]⁺=208.1); ¹H NMR (400 MHz, DMSO-d₆) δ=8.83 (br s, 3H), 7.50-7.42 (m, 2H), 7.41-7.32 (m, 2H), 3.03 (br dd, J=2.4, 14.0 Hz, 1H), 2.45-2.27 (m, 2H), 2.21-2.05 (m, 1H), 1.97 (td, J=2.8, 9.6 Hz, 1H), 1.81 (br d, J=11.6 Hz, 1H), 1.71-1.47 (m, 2H); ¹³C NMR (101 MHz, DMSO-d₆) δ=206.52, 164.22, 161.76, 130.78, 130.69, 130.08, 130.05, 116.90, 116.68, 66.26, 34.75, 27.52, 21.53; ENT-2 HC, RT=2.945 mi (assigned here as the R isomer, 1R); LCMS (R_{T}=1.449 min, MS calc.: 207.1, [M+H]⁺=208.0); ¹H NMR (400 MHz, DMSO-d₆) δ=8.84 (br s, 3H), 7.49-7.42 (m, 2H), 7.40-7.33 (m, 2H), 3.03 (br dd, J=1.6, 14.0 Hz, 1H), 2.45-2.27 (m, 2H), 2.23-2.06 (m, 1H), 1.97 (dt, J=2.8, 6.1 Hz, 1H), 1.81 (br d, J=11.6 Hz, 1H), 1.70-1.46 (m, 2H); ¹³C NMR (101 MHz, DMSO-d₆) δ=206.50, 164.22, 161.76, 130.78, 130.70, 130.08, 130.05, 116.89, 116.68, 66.26, 34.75, 27.51, 21.52.

The retention times above, which identify the enantiomers, were determined using the free bases using the following chiral analytical method: column: Chiralpak AD-3 (150 mm×4.6 mm I.D, 3 μm); mobile phase: A: CO₂ B: EtOH (0.1% IPAm, v/v); gradient (Time (min)/A %/B %): 0.0/90/10, 0.5/90/10, 3.5/50/50, 4.5/50/50, 5.0/90/10; flow rate: 2.5 mL/min; column temp.: 35° C.; ABPR: 2,000 psi.

### Step 4: Preparation of (S)-2-(4-fluorophenyl)-2-(methylamino)cyclohexan-1-one (2S) and (R)-2-(4-fluorophenyl)-2(methylamino)cyclohexan-1-one (2R)

Compound 1S_FB (540 mg, 2.61 mmol, 1 eq) and methyl trifluoromethanesulfonate (427.59 mg, 2.61 mmol, 285.06 μL, 1 eq) were combined in hexafluoroisopropanol (40 mL) at 0° C. under N₂ atmosphere and then the mixture was allowed to warm to 25° C. and stirred for 12 h. On completion, the residue was adjusted to pH 7 with sat. aq. Na₂CO₃ (10 mL) and the combined organic phase was washed with brine (100 mL*2), dried over Na₂SO₄, filtered, and concentrated in vacuum. The residue was purified by prep-HPLC (column: Waters Xbridge C18 (150 mm*50 mm, 10 μm); mobile phase: A: water (10 mM NH₄HCO₃), B: ACN; B %: 30%-50%, 10 min) to afford 2S (260 mg, 1.18 mmol, 45.10% yield) as a white solid. Compound 2R was prepared by the same procedure starting from 1R_FB (590 mg 2.85 mmol) in hexafluoroisopropanol (60 mL) (other quantities scaled based on molar equivalents) and obtained as an off-white solid (260 mg, 1.18 mmol, 41.27% yield).

2S (assigned here as the S isomer) (free base); LCMS (R_{T}=1427 min, MS calc.: 221.1, [M+H]⁺=222.1); ¹H NMR (400 MHz, CHLOROFORM-d) δ=7.21 (dd, J=5.4, 8.8 Hz, 2H), 7.10-7.02 (m, 2H), 2.85-2.74 (m, 1H), 2.49-2.37 (m, 1H) 2.36-2.25 (m, 1H) 2.22 (br s, 1H), 2.03 (s, 3H), 1.96 (dt, J=3.2, 5.8 Hz, 1H), 1.88-1.64 (m, 4H); ¹³C NMR (101 MHz, CHLOROFORM-d) δ=211.25, 163.22, 160.76, 134.80, 134.77, 128.98, 128.90, 115.80, 115.59, 69.38, 39.73, 35.92, 28.92, 27.72, 22.24; 2R (assigned here as the R isomer) (free base); LCMS (R_{T} 1.415 min, MS calc: 221.1, [M+H]⁺=: 222.1); ¹H NMR (400 MHz, CHLOROFORM-d) δ=7.25-7.17 (m, 2H), 7.11-7.02 (m, 2H), 2.85-2.75 (m, 1H), 2.48-2.38 (m 1H), 2.35-2.19 (m, 2H), 2.04 (s, 3H), 1.97 (br dd, J=2.8, 6.1 Hz, 1H), 1.89-1.66 (m, 4H); ¹³C NMR (101 MHz, CHLOROFORM-d) δ=211.24, 163.22, 160.77, 134.78, 134.74, 128.99, 128.91, 115.81, 115.60, 69.38, 39.73, 35.91, 28.91, 27.72, 22.24.

### Example 2: Preparation of Compound 3 and its Enantiomers

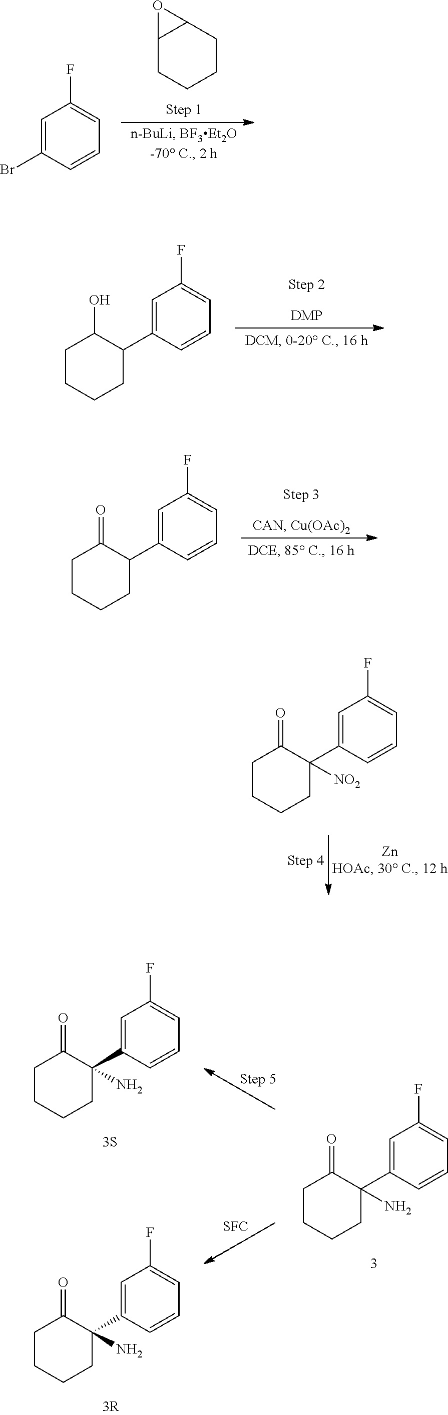

### Step 1: Preparation of 2-(3-fluorophenyl)cyclohexan-1-ol

To a solution of 1-bromo-3-fluorobenzene (10 g, 57.14 mmol, 6.37 mL, 1 eq) in T-F (150 mL) was added n-BuLi (2.5 M, 25.14 mL, 1.1 eq) dropwise at −70° C. under N₂. After addition, the mixture was stirred at −70° C. for 0.5 h. Then, 7-oxabicyclo[4.1.0]heptane (6.17 g, 62.86 mmol, 6.36 mL, 1.1 eq) and BF₃·Et₂O (18.98 g, 62.86 mmol, 25.1 mL, 1.1 eq) were added dropwise at −70° C. The resulting mixture was stirred at −70° C. for 1.5 h. On completion, the reaction was carefully quenched with aq. NH₄Cl (800 mL) and then extracted with EA (300 mL×3). The combined organic phase was washed with brine (500 mL), dried over Na₂SO₄, filtered, and concentrated under reduced pressure to give a residue. The residue was purified by column chromatography (SiO₂, petroleum ether/ethyl acetate=50/1 to 30/1) to afford 2-(3-fluorophenyl)cyclohexan-1-ol (7 g, 36.04 mmol, 63.06% yield) as a colorless oil. ¹H NMR (400 MHz, CHLOROFORM-d) δ=7.57-7.47 (m, 1H), 7.26 (d, J=7.6 Hz, 1H), 7.23-7.12 (m, 2H), 3.86 (dt, J=4.4, 10.1 Hz, 1H), 2.73-2.61 (m, 1H), 2.41-2.28 (m, 1H), 2.12-2.06 (m, 2H), 2.03-1.96 (m, 1H), 1.72-1.49 (m, 4H).

### Step 2: Preparation of 2-(3-fluorophenyl)cyclohexan-1-one

To a solution of 2-(3-fluorophenyl)cyclohexan-1-ol (6.7 g, 34.49 mmol, 1 eq) in DCM (70 mL) was added DMP (43.89 g, 103.48 mmol 32.04 mL 3 eq) dropwise at 0° C. under N₂. The mixture was then allowed to warm to 20° C. and stirred for 16 h. On completion, the mixture was filtered and the filtrate was washed with sat. aq. Na₂SO₃ (300 mL). Then the mixture was adjusted to pH 8 with aq. NaHCO₃ (100 mL) and the mixture was extracted with DCM (50 mL×3). The combined organic phase was dried over Na₂SO₄, filtered, and concentrated under vacuum. The residue was purified by column chromatography (SiO₂, petroleum ether/ethyl acetate=50/1 to 40/1) to afford 2-(3-fluorophenyl)cyclohexan-1-one (4.4 g, 22.89 mmol, 66.36% yield) as a yellow oil. ¹H NMR (400 MHz, CHLOROFORM-d) δ=7.33-7.26 (m, 1H), 6.99-6.84 (m, 3H), 3.62 (dd, J=5.6, 12.1 Hz, 1H), 2.58-2.40 (m, 2H), 2.34-2.23 (m, 1H), 2.22-2.11 (m, 1H), 2.07-1.93 (m, 2H), 1.90-1.77 (m, 2H).

### Step 3: Preparation of 2-(3-fluorophenyl)-2-nitrocyclohexan-1-one

A mixture of 2-(3-fluorophenyl)cyclohexan-1-one (4.2 g, 21.85 mmol, 1 eq), Cu(OAc)₂ (793.69 mg, 4.37 mmol, 0.2 eq), and CAN (23.96 g, 43.70 mmol, 21.78 mL, 2 eq) in DCE (40 mL) was degassed and purged with N₂ 3 times and then stirred at 85° C. for 16 h under N₂ atmosphere. On completion, the reaction mixture was filtered and the filtrate was concentrated. The residue was purified by column chromatography (SiO₂, petroleum ether/ethyl acetate=50/1 to 0/1) to afford 2-(3-fluorophenyl)-2-nitrocyclohexan-1-one (2.42 g, 10.20 mmol, 46.69% yield) as a yellow oil. ¹H NMR (400 MHz, CHLOROFORM-d) δ=7.50-7.40 (m, 1H), 7.23-7.11 (m, 2H), 7.11-7.04 (m, 1H), 3.16-3.05 (m, 1H), 2.84-2.66 (m, 2H), 2.62-2.51 (m, 1H), 2.06-1.86 (m, 3H) 1.86-1.73 (m, 1H).

### Step 4: Preparation of 2-amino-2-(3-fluorophenyl)cyclohexan-1-one 3

To a mixture of 2-(3-fluorophenyl)-2-nitrocyclohexan-1-one (1.99 g, 8.39 mmol, eq) in AcOH (20 mL) was added Zn (8.23 g, 125.83 mmol, 15 eq) in several portions and the resulting mixture was stirred at 30° C. for 12 h. On completion, the reaction mixture was filtered and the filtrate was concentrated. The residue was adjusted to pH 8 with aq. NaHCO₃ (40 mL) and the aqueous phase was extracted with DCM (50 mL×2). The combined organic phase was dried over Na₂SO₄, filtered, and concentrated under vacuum. The residue was purified by prep-HPLC (column: Welch Xtimate C18 250*70 mm, 10 μm; mobile phase: A: water (10 mM NH₄HCO₃), B: ACN; B %: 15%-45%, 20 min) to afford 2-amino-2-(3-fluorophenyl)cyclohexan-1-one (1.11 g, 5.36 mmol 63.85% yield, 3) as a yellow oil. ¹H NMR (400 MHz, CHLOROFORM-d) δ=7.38-7.31 (m, 1H), 7.04-6.95 (m, 3H), 2.84-2.72 (m, 1H), 2.54-2.43 (m, 1H), 2.42-2.31 (m, 1H), 2.07-1.96 (m, 1H), 1.85-1.61 (m, 4H).

Note: The free base of this compound is unstable and dimerizes over time. It should be stored frozen or quickly converted to the HCl salt to prevent this.

### Step 5: Preparation of (S)-2-amino-2-3-fluorophenyl)cyclohexan-1-one (3S and (R)-2-amino-2-(3-fluorophenyl)cyclohexan-1-one (3R)

The racemate 3 (1.11 g, 5.36 mmol) was separated by SFC (column: DAICEL CHIRALPAK AD (250 mm*30 mm 10 μm); mobile phase: A: CO₂, B: 0.1% NH₃H₂O in ETOH; B %: 30%, multi-injection process with 5-min spacing between injections). To the eluate containing each separated enantiomer was added 1M aq. HCl to adjust the pH-J to 4-5 and then each mixture was concentrated under vacuum to provide crude 3S (RT=2.081 min, 376.4 mg, HCl salt) and crude 3R (RT=2.791 min, 437 mg, HCl salt) as white solids. However, both materials were contaminated by NH₄Cl, so the following procedure was conducted to remove NH₄Cl. Each crude enantiomer HCl was re-dissolved in DCM (10 mL), the pH was adjusted to 9-10, and the organic phase was washed with H₂O (5 mL*3). The organic phase was then concentrated under vacuum, 1 mL CH₃CN and 10 mL H₂O was added to residue, and then the pH was adjusted to 4-5 with 1M aq. HCl. Then the mixture was lyophilized to provide the HC salts of the pure enantiomers 3S (RT=2.081 min, 330 mg, 1.18 mmol, HCl salt) and 3R (RT=2.791 min, 320 mg, 1.14 mmol, HCl salt) as white solids.

3S, RT=2.081 min (assigned here as the S isomer) (HCl salt); LCMS (R_{T}=2.298 min, MS calc.: 207.1, [M+H]⁺=208.0), ¹H NMR (400 MHz, DMSO-d₆) δ=8.63 (br s, 31H), 7.61-7.53 (m, 1H), 7.38-7.26 (m, 2H), 7.22-7.16 (m, 1H), 3.00 (br dd, J=2.0, 14.0 Hz, 1H), 2.49-2.29 (m, 2H), 2.12 (dt, J=3.6, 13.4 Hz, 1H), 2.03-1.92 (m, 1H), 1.82 (br d, J=10.4 Hz, 1H), 1.71-1.49 (m, 2H); ¹³C NMR (101 MHz DMSO-d₆) δ=206.56, 164.08, 161.65, 137.13, 137.06, 132.01, 131.93, 124.43, 124.40, 117.18, 116.97, 115.32, 115.09, 66.39, 34.94, 27.41, 21.63; 3R, RT=2.791 in (assigned here as the R isomer) (HCl salt); LCMS (R_{T}=0.634 min, MS calc.: 207.1, [M+H]⁺=208.1); ¹H NMR (400 MHz, DMSO-d₆) δ=7.99 (br s, 3H), 70.58-7.49 (m, 1H), 7.36-7.23 (m, 2H), 7.20-7.16 (m, 1H), 2.92 (br d, J=14.0 Hz, 1H), 2.49-2.41 (m, 11H), 2.40-2.28 (m, 1H), 2.12-2.01 (m, 1H). 1.96 (dt, J=2.8, 6.2 Hz, 1H), 1.87-1.78 (m, 1H), 1.71-1.49 (m, 2H); ¹³C NMR (101 MHz, DMSO-d₆) δ=206.56, 164.08, 161.65, 137.13, 137.06, 132.01, 131.93, 124.43, 124.40, 117.18, 116.97, 115.32, 115.09, 66.39, 34.94, 27.41, 21.63.

The retention times above, which identify the enantiomers, were determined using the free bases using the following chiral analytical method: column: Chiralpak AD-3 (150 mm×4.6 mm I.D., 3 μm); mobile phase: A: CO₂ B: EtOH (0.1% IPAm, v/v); gradient (Time (min)/A %/B %): 0.0/90/10, 0.5/90/10, 3.5/50/50, 4.5/50/50, 5.0/90/10; flow rate: 2.5 mL/min; column temp.: 35° C.; ABPR: 2,000 psi.

### Example 3: Preparation of Compounds 10 and 11

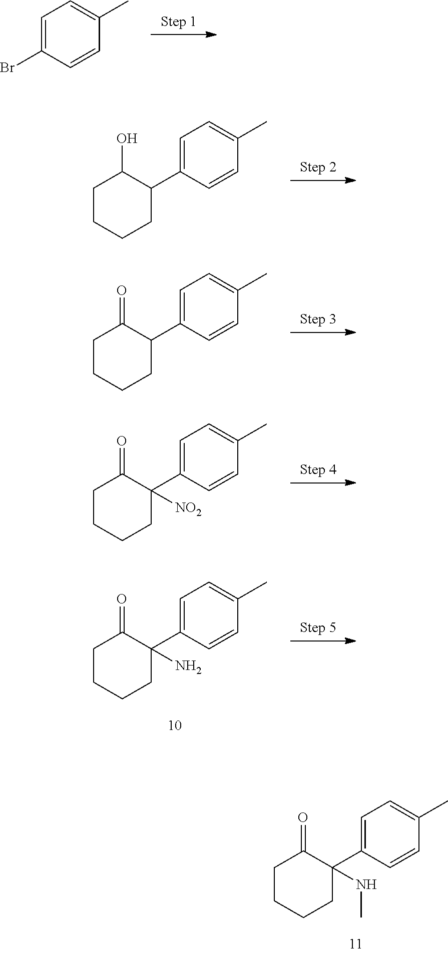

### Step 1: Preparation of 2-(p-tolyl)cyclohexan-1-ol

To a solution of 1-bromo-4-methyl-benzene (15 g, 87.70 mmol, 10.79 mL, 1 eq) in THF (200 mL) was cooled to −70° C. Then n-BuLi (2.5 M, 38.59 mL, 1.1 eq) was added. The mixture was stirred at −70° C. for 0.5 h and then 7-oxabicyclo[4.1.0]heptane (9.47 g, 96.47 mmol, 9.76 mL, 1.1 eq) and BF₃·Et₂O (13.69 g, 96.47 mmol, 11.91 mL, 1.1 eq) were added. The mixture was stirred at −70° C. for 1.5 hrs. On completion, the reaction was quenched with sat. aq. NH₄Cl (40 mL) slowly and then extracted with EtOAc (50 mL×3). The combined organic phase was washed with brine (50 mL), dried over Na₂SO₄, filtered, and concentrated in vacuo. The residue was purified by silica gel chromatography (Petroleum ether/Ethyl acetate=100/1, 5/1) to afford 2-(p-tolyl)cyclohexan-1-ol (13 g, 68.32 mmol, 77.9% yield) as a white solid. ¹H NMR (400 MHz, CHLOROFORM-d) δ=7.18-7.13 (m, 4H), 3.69-3.61 (m, 1H), 2.44-2.37 (m, 1H), 2.35 (s, 3H), 2.16-2.09 (m, 1H), 1.91-1.82 (m, 2H), 1.80-1.73 (m, 1H), 1.55-1.31 (m, 4H).

### Step 2: Preparation of 2-(p-tolyl)cyclohexan-1-one

To a mixture of 2-(p-tolyl)cyclohexan-1-ol (13 g, 68.32 mmol, 1 eq) in CH₂Cl₂ (50 mL) was added Dess-Martin Periodinane (43.47 g, 102.48 mmol, 31.73 mL, 1.5 eq) in several portions at 0° C. (maintaining the temperature at 0° C. during addition). Then the mixture was stirred at 20° C. for 12 h. The mixture was filtered. The filtrate was washed with sat. aq. Na₂SO₃, sat. aq. Na₂CO₃, and brine, dried over Na₂SO₄ filtered, and concentrated. The residue was purified by silica gel chromatography (PE:EA=50:1-5:1) to afford 2-(p-tolyl)cyclohexan-1-one (12.01 g, 63.82 mmol, 93.41% yield) as a while solid. ¹H NMR (400 MHz, CHLOROFORM-d) δ=7.20-7.13 (m, 2H), 7.08-7.02 (m, 2H), 3.63-3.55 (m, 1H), 2.58-2.42 (m, 21H), 2.35 (s, 3H), 2.32-2.23 (m, 11H), 2.20-2.12 (m, 11H), 2.08-1.98 (m, 2H), 1.90-1.81 (m, 2H).

### Step 3: Preparation of 2-nitro-2-(p-tolyl)cyclohexan-1-one

A mixture of 2-(p-tolyl)cyclohexan-1-one (11 g, 58.43 mmol, 1 eq), ceric ammonium nitrate (CAN, 64.06 g, 116.86 mmol, 58.24 mL, 2 eq), and Cu(OAc)₂ (2.12 g, 11.69 mmol, 0.2 eq) in DCE (150 mL) was stirred at 85° C. for 12 h. The reaction mixture was cooled, filtered, and the filtrate was concentrated. The residue was purified by column chromatography (SiO₂, PE/EA:=1/0 to 0/1) to afford 2-nitro-2-(p-tolyl)cyclohexan-1-one (5.98 g, 25.64 mmol, 43.88% yield) as a yellow oil, ¹H NMR (400 MHz, CHLOROFORM-d) δ=7.36-7.27 (m, 4H), 3.10 (ddd, J=3.6, 10.9, 14.4 Hz, 1H), 2.99-2.89 (m, 1H), 2.76-2.65 (m, 1H), 2.65-2.54 (m, 1H), 2.44 (s, 3H), 2.05-1.92 (m, 3H), 1.86-1.73 (m, 1H).

### Step 4: Preparation of 2-amino-2-(p-toyl)cyclohexan-1-one (10)

To a solution of 2-nitro-2-(p-tolyl)cyclohexan-1-one (4.98 g, 21.35 mmol, 1 eq) in AcOH (40 mL) was added Zn (33.50 g, 512.38 mmol, 24 eq) at 0° C. The mixture was stirred at 25° C. for 12 h. On completion, the mixture was filtered and concentrated. The residue was adjusted to pH=7 with aq. Na₂CO₃ solution (150 mL). The aqueous phase was extracted with DCM (200 mL×2) and the combined organics were dried over anhydrous Na₂SO₄, filtered, and concentrated in vacuo. The residue was purified by column chromatography (SiO₂, PE/EA=1/0 to 0/1) to afford 2-amino-2-(p-tolyl)cyclohexan-1-one (1.3 g, 6.40 mmol, 29.95% yield) (10) as a yellow oil. LCMS (R_{T}=1.618 min, MS calc.: 203.3, [M+H]⁺=204.1); ¹H NMR (400 MHz, CHLOROFORM-d) δ=7.14 (q, J=8.4 Hz, 4H), 2.90-2.75 (m, 1H), 2.48-2.35 (m, 2H), 2.32 (s, 3H), 1.96 (br s, 3H), 1.83-1.52 (m, 4H); ¹³C NMR (101 MHz, CHLOROFORM-d) δ=213.76, 138.92, 137.49, 129.93, 126.04, 66.28, 39.83, 39.53, 28.22, 22.76, 20.99.

### Step 5: Preparation of 2-(methylamino)-2-(p-tolyl)cyclohexan-1-one 11)

A mixture of 2-amino-2-(p-tolyl)cyclohexan-1-one (583 mg, 2.87 mmol, 1 eq) in hexafluoroisopropanol (HFIP, 60 mL) was added methyl trifluoromethanesulfonate (470.65 mg, 2.87 mmol, 313.76 uL, 1 eq) at 0° C. Then the mixture was stirred at 25° C. for 12 h under N₂ atmosphere. The mixture was filtered and concentrated. The residue was adjusted to pH=7 with sat. aq. Na₂CO₃ solution (100 mL). The aqueous phase was extracted with BA (100 mL×2). The combined organic phase was washed with brine (100 mL×1), dried with anhydrous Na₂SO₄, filtered, and concentrated in vacuo. The residue was purified by prep-HPLC (column: Welch Xtimate C18 250*70 mm, 10 μm; mobile phase: A: water (0.05% NH₃H₂O), B: ACN; B %: 10%-45%, 35 min) to afford 2-(methylamino)-2-(p-tolyl)cyclohexan-1-one (398.86 mg, 1.84 mmol, 64.00% yield) (11) as a yellow oil. LCMS (R_{T}=1.574 min, MS calc.: 217.3, [M+H]⁺=218.1): ¹H NMR (400 MHz, CHLOROFORM-d) δ=7.21-7.17 (m, 2H), 7.16-7.10 (m, 2H), 2.92-2.83 (m, 1H), 2.44-2.36 (m, 2H), 2.35 (s, 3H), 2.04 (s, 3H), 2.01-1.91 (m, 1H), 1.86-1.68 (m, 4H); ¹³C NMR (101 MHz, CHLOROFORM-d) δ=211.35, 137.45, 129.60, 127.17, 69.80, 39.76, 35.30, 28.87, 27.78, 22.31, 21.04.

### Example 4: Preparation of Compound 12

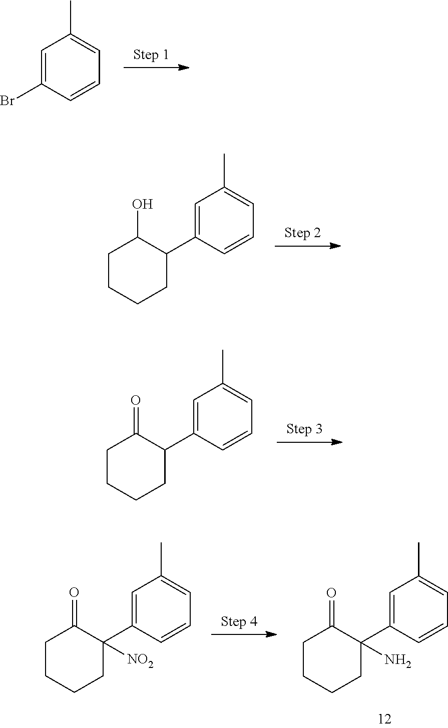

### Step 1: Preparation of 2-(m-tolyl)cyclohexan-1-ol

A mixture of 1-bromo-3-methyl-benzene (15 g, 87.70 mmol, 10.64 mL, 1 eq) in THF (150 mL) was cooled to −70° C. Then n-BuLi (2.5 M, 38.59 mL, 11 eq) was added. The mixture was stirred at −70° C. for 0.5 hr and then 7-oxabicyclo[4.1.0]heptane (9.47 g, 96.47 mmol, 9.76 mL, 1.1 eq) and BF₃·Et₂O (13.69 g, 96.47 mmol, 11.91 mL, 1.1 eq) were added. The mixture was stirred at −70° C. for 1.5 h. The mixture was poured into sat. aq. NH₄Cl (200 mL) and extracted with EA (100 mL×2). The organic layer was washed with brine, dried over Na₂SO₄, filtered, and concentrated. The residue was purified by silica gel (PE:EA=100:1-10:1) to afford 2-(m-tolyl)cyclohexan-1-ol (13 g, 68.32 mmol, 77.9% yield) as a colorless oil. ¹H NMR (400 MHz, CHLOROFORM-d) δ=7.33-7.28 (m, 1H), 7.16-7.09 (m, 3H), 3.72 (dt, J=4.0, 10.0 Hz, 1H), 2.50-2.44 (m, 1H), 2.41 (m, 3H), 2.22-2.14 (m, 1H), 1.92 (br d, J=10.8 Hz, 2H), 1.83 (br d, J=12.4 Hz, 1H), 1.61-1.36 (m, 4H).

### Step 2: Preparation of 2-(m-tolyl)cyclohexan-1-one

To a mixture of 2-(m-tolyl)cyclohexan-1-ol (13 g, 68.32 mmol, 1 eq) in DCM (50 mL) was added Dess-Martin Periodinane (43.47 g, 102.48 mmol, 31.73 mL, 1.5 eq) in several portions at 0° C. (maintaining the temperature at 0° C. during addition). Then the mixture was stirred at 20° C. for 12 h. The mixture was filtered and the filtrate was washed with sat. aq. Na₂SO₃, sat. aq. Na₂CO₃, and brine, dried over Na₂SO₄, filtered, and concentrated. The residue was purified by silica gel chromatography (PE:EA=1:0-5:1) to afford 2-(m-tolyl)cyclohexan-1-one (13 g, crude) as a white solid. ¹H NMR (400 MHz, CHLOROFORM-d) δ 7.26-7.21 (m, 1H) 7.10-7.06 (m, 1H), 6.98-6.93 (m, 2H), 3.62-3.55 (m, 1H), 2.58-2.44 (m, 2H) 2.35 (s, 3H), 2.31-2.23 (m, 1H), 2.21-2.13 (m, 1H), 2.08-1.97 (m, 2H), 1.90-1.83 (m, 2H).

### Step 3: Preparation of 2-(n-tolyl)-2-nitro-cyclohexan-1-one

A mixture of 2-(m-tolyl)cyclohexan-1-one (11 g, 58.43 mmol, 1 eq), ceric ammonium nitrate (CAN, 64.06 g, 116.86 mmol, 58.24 mL, 2 eq), and Cu(OAc)₂ (2.12 g, 11.69 mmol, 0.2 eq) in DCE (200 ml) was stirred at 85° C. for 12 h. The mixture was cooled and filtered and the filter cake was washed by EtOAc (80 mL×4). The filtrate was concentrated under vacuum to give a residue that was purified by silica gel chromatography (SiO₂, PE/EtOAc=10/1) to afford 2-(m-tolyl)-2-nitro-cyclohexan-1-one (3 g, 12.86 mmol, 22.01% yield) as a yellow oil. ¹H NMR (400 MHz, CHLOROFORM-d) δ=7.39-7.33 (m, 1H), 7.28 (br s, 1H), 7.18-7.13 (m, 2H), 3.06 (ddd, J=3.2, 10.7, 14.3 Hz, 1H), 2.96-2.86 (m, 11H), 2.74-2.64 (m, 1H), 2.62-2.52 (m, 1H), 2.40 (s, 3H), 1.99-1.88 (m, 3H), 1.78 (ddd, J=3.6, 6.6, 10.4 Hz, 1H).

### Step 4: Preparation of 2-amino-2-(m-tolyl)cyclohexan-1-one 12)

To a mixture of 2-(m-tolyl)-2-nitro-cyclohexan-1-one (2.5 g, 10.72 mmol, 1 eq) in AcOH (30 mL) was added Zn (16.82 g, 257.22 mmol, 24 eq) over 1 h and the mixture was then stirred at 20° C. for 12 h. On completion, the mixture was filtered and the filtrate was concentrated. The residue was dissolved with DCM (10 mL), adjusted to pH=8 with sat. Na₂CO₃, and extracted with DCM (10 mL×2). The organic phase was dried over Na₂SO₄, filtered, aid concentrated to afford 2-amino-2-(m-tolyl)cyclohexan-1-one (1.90 g, 9.35 mmol, 87.21% yield) (12) as a yellow oil. LCMS (R_{T}=1.629 min, MS calc.: 203.3, [M+H]⁺=204.1); ¹H NMR (400 MHz, CHLOROFORM-d) δ=7.30-7.27 (m, 1H), 7.11 (d, J=7.6 Hz, 1H), 7.09-7.05 (m, 2H), 2.91-2.83 (m, 1H), 2.49-2.41 (m, 2H), 2.36 (s, 3H), 2.07-1.94 (m, 1H), 1.80-1.650 (m, 4H); ¹³C NMR (101 MHz, CHLOROFORM-d) δ=213.83, 141.84, 139.01, 129.14, 128.46, 126 79, 123.08, 66.50, 39.94, 39.49, 28.24, 22.78, 21.57.

### Example 5. Preparation of Compound 7R

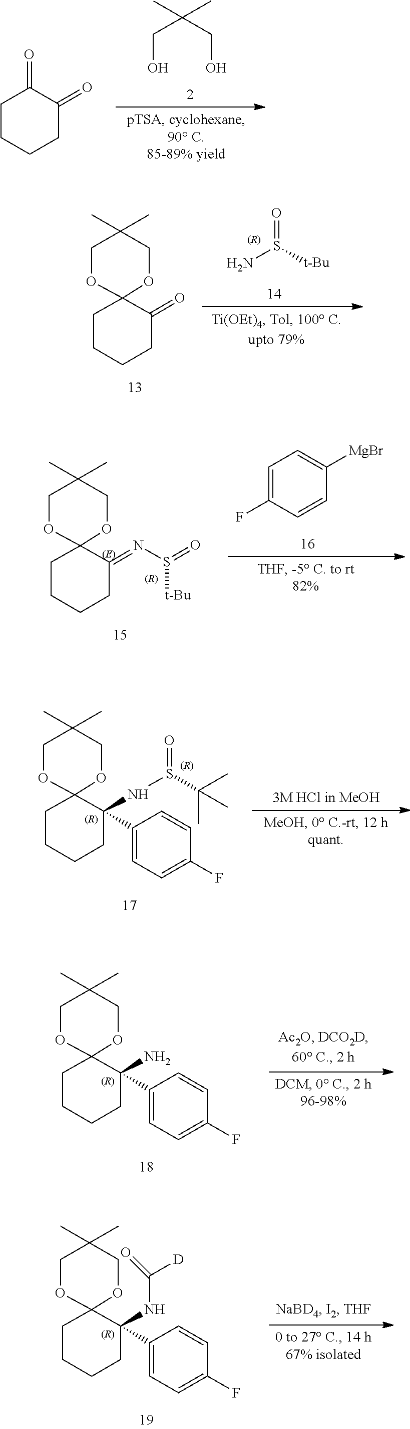

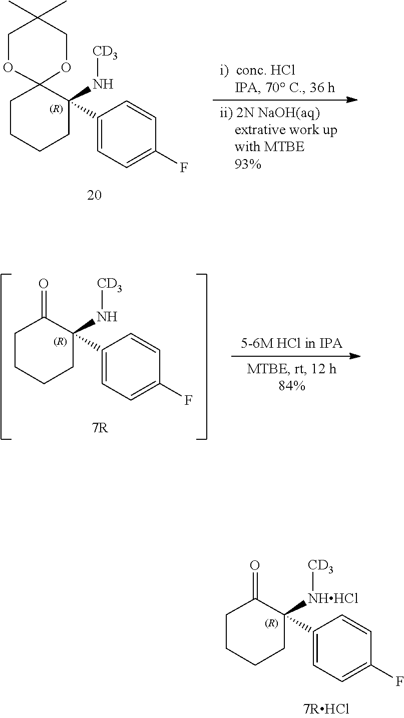

### Procedure for the Preparation of 13

A 1,000 mL jacketed reactor equipped with an over-head stirrer and a Dean-Stark apparatus was charged with 1,2-cyclohexanedione (50.0 g, 428 mmol), 2,2-dimethylpropane-1,3-diol (54.0 g, 514 mmol), p-TSA (1.66 g, 8.6 mmol), and cyclohexane (200 mL, 4 V), and the resulting suspension was heated at reflux (80° C.) for 3 h to obtain a complete conversion of the starting material. It was then cooled to 20° C. and charged with 1N NaOH(aq) (1 V) followed by MTBE (2 V) and stirred. The phases were separated, and the aqueous phase was further extracted with MTBE (2×2V) and the combined organics were washed once with 10% brine (5 V) and concentrated. The mixture was azeotroped once with 1 V of toluene to obtain 113 g of 13 (Q-NMR assay: 66%, yield 87.6%). The crude product was taken to the next step without further purification. ¹H NMR (400 MHz, CDCl₃) δ 3.55 (d, J=11.1 Hz, 1H), 3.32 (d, J=11.1 Hz, 1H), 2.38-2.35 (m, 2H), 1.8-1.79 (m, 2H), 1.67-1.63 (m, 4H), 1.06 (s, 3H), 0.55 (s, 3H).

### Procedure for the Preparation of 15

A 1 L round bottom flask equipped with an overhead stirrer was charged with compound 13 (33.3 g, 60% wt. %, 0.101 mol), (R)-t-Bu-Sulfinamide (14, 14.62 g, 0.121 mol), toluene (80 ml), and Ti(OEt₄) (25.31 mL, 0,121 mol), at room temperature. The mixture was heated at 80° C. for 5-6 h followed by cooling to room temperature to obtain a dark solution. To this solution was added EDTE (47.5 g, 2 equiv.) and the mixture was heated at 55° C. for 60 minutes followed by cooling to room temperature. To the above solution at 25-28° C. was added 12% NaCl (aq) (5 V) and the mixture was stirred for about 5 mins and allowed to settle. The phases were separated and the aqueous phase was re-extracted twice with toluene (5 V). The combined organics were washed once with water. The organic phase was filtered through a plug of activated charcoal (6%) and SiO₂ (10%) and concentrated to obtain the crude product as a yellow-orange semi-solid (18.9 g net product by NMR wt %, 63%). The product crystalized out as off-white solid upon standing, which was filtered and carried to the next step. ¹H NMR (400 MHz, CDCl₃) δ 3.83 (d, J=11.0 Hz, 1H), 3.72 (d, J=11.0 Hz, 1H), 3.44-3.38 (m, 2H), 3.13-3.07 (m, 1H), 2.89-2.83 (m, 1H), 1.98-1.85 (m, 1H), 1.81-1.71 (m, 1H), 1.31 (s, 9H), 1.21 (s, 3H), 0.72 (s, 3H).

### Procedure for the Preparation of 17

To a stirred solution of compound 15 (17.5 g, 58.0 mmol) in THF (70 mL) at −5° C. was added a 1 M solution of 4-F-phenylmagnesium bromide in THF (16, 116 mL, 116 mmol, 2 equiv.) dropwise. The resulting reaction mixture was stirred at −5° C. for 4 h and then at room temperature for 14 h. TLC (50% EtOAc/hexnanes) indicated the complete conversion of the starting material. The reaction mixture was then cooled to 0° C. and saturated aqueous NH₄Cl (70 ml) was added dropwise. After warming to room temperature, the aqueous phase was extracted with MTBE (2×35 mL) and the combined organic layer was washed with water and dried over Na₂SO₄. Evaporation of the solvent gave the crude product, which was re-slurried with heptane followed by filtration to give compound 17 (18.24 g, 79%) as a white solid. ¹H NMR (400 MHz, CDCl₃) δ 7.72-7.68 (m, 2H), 6.98-6.94 (m, 2H), 4.51 (s, 1H), 3.67-3.60 (m, 2H), 3.35 (dd, J=11.3 Hz and 2.6 Hz, 1H), 3.27 (dd, J=11.3 Hz and 2.5 Hz, 1H), 2.70-2.63 (m, 1H), 2.33-2.27 (m, 1H), 2.05-2.01 (m, 1H), 1.98-1.88 (m, 1H), 1.76-1.66 (m, 11H), 1.62-1.45 (m, 2H), 1.17 (s, 9H), 0.84 (s, 3H), 0.69 (s, 3H); ¹³C NMR (101 MHz, CDCl₃) δ 161.9 (d, J_{C-F}=246.9 Hz), 136.64 (d, J_{C-F}=2.93 Hz), 132.5 (d, J_{C-F}=7.68 Hz), 113.4 (d, J_{C-F}=20.76 Hz), 99.8, 69.9, 69.5, 65.8, 56.3, 34.4, 29.7, 22.9, 22.87, 22.81, 22.1, 21.4; ¹⁹F NMR (376 MHz, CDCl₃) δ-116.3.

### Procedure for the Preparation of 18

To a suspension of compound 17 (45.0 g, 113 mmol) in methanol (180 mL) at 0° C. was added a solution of 3 M HCl in methanol (113 mL, 339 mmol, 3 equiv.) dropwise. The resulting reaction mixture was allowed to warm to room temperature and stirred for 12-14 h. After completion of the reaction, the mixture was cooled to 0° C. and saturated aqueous NaHCO₃ (225 mL) was added dropwise. To the resulting suspension, CH₂Cl₂ (90 mL) was added to dissolve the product and the phases were separated. The aqueous phase was extracted with CH₂Cl₂ (2×90 mL) and the combined organics were washed with brine, dried (Na₂SO₄), and concentrated to afford crude compound 18 (27.1 g, 82% quant) as a white solid, which was carried to the next step without further purification. ¹H NMR (400 MHz, CDCl₃) δ 7.62-0.52 (m, 2H), 6.99-6.90 (m, 2H), 3.57 (dd, J=23.4, 11.4 Hz, 2H), 3.16 (ddd, J=11.2, 8.4, 2.7 Hz, 2H), 2.53-2.36 (m, 2H), 1.86-1.34 (m, 8H), 0.59 (s, 3H), 0.36 (s, 3H); ¹³C NMR (101 MHz, CDCl₃) δ 162.8, 160.4, 141.5, 130.3, 130.2, 113.4, 113.2, 99.6, 70.0, 69.9, 60.4, 34.8, 29.8, 22.5, 22.3, 22.2, 22.1, 21.2; ¹⁹F NMR (376 MHz, CDCl₃) δ-118.5.

### Procedure for the Preparation of 19

A mixture of acetic anhydride (1.9 mL, 13.63 mmol) and formic acid-cd (0.54 mL, 13.63 mmol) was stirred at 60° C. for 2 h followed by gradually cooling to 0° C. To the above mixture at 0° C. was then added a solution of compound 18 (1.0 g, 3.41 mmol) in CH₂Cl₂ (5 mL) and the mixture was allowed to stir at 0° C. for 2 h. TLC (30% EtOAc/hexnanes) indicated the complete conversion of the starting material. The mixture was then neutralized by slow addition of an aqueous solution of sodium bicarbonate (Caution: gas evolution) and extracted with CH₂Cl₂. The combined organics were washed once with satd. NaHCO₃ (aq) and water, followed by brine, dried (Na₂SO₄), and concentrated to obtain the crude 19 (1.1 g, quantitative) as an off-white solid, which was carried to the next step without further purification. ¹H NMR (400 MHz, CDCl₃) δ 7.50-7.36 (m, 2H), 7.02-6.88 (m, 2H), 6.56-6.11 (m, 1H), 3.66-3.49 (m, 2H), 3.26-3.11 (m, 2H), 2.98-2.87 (m, 1H), 2.71-2.52 (m, 2H), 2.42-2.29 (m, 1H), 2.11-2.00 (m, 1H), 1.71-1.32 (m, 4H), 0.62-0.57 (m, 3H), 0.33-0.23 (m, 3H); ¹³C NMR (101 MHz, CDCl₃) δ 163.3, 163.2, 160.9, 160.8, 160.1, 138.6 (2C), 136.1, 136.0, 131.1, 131.0, 130.5, 130.4, 114.0, 113.8, 113.7, 113.5, 98.0, 97.9, 70.1, 70.0 (2C), 69.9, 65.4, 63.8, 32.5, 29.9 (2C), 29.5, 23.7, 23.1, 22.1 (2C), 21.9 (C), 21.8, 21.2, 20.5; ¹⁹F NMR (376 MHz, CDCl₃) δ-116.6, -117.7.

### Procedure for the Preparation of 20

To a stirring suspension of 19 (1.1 g, 3.42 mmol) and NaBD₄ (572 mg, 13.66 mmol) in THF (4 mL) at 0° C. was added a solution of iodine (1.13 g, 4.44 mmol) in THF (2 mL) drop-wise. The mixture was then allowed to warm to room temperature for 14 h. The mixture, was then cooled to 0° C. and quenched with slow addition of MeOH (2 mL) followed by heating at 40° C. for 1 h. The resulted clear solution was then concentrated and treated with MTBE followed by water and 1N NaOH(aq) to obtain clear phase separation. The MTBE later was separated and the aqueous phase was further extracted once with MTBE. The combined organics were then washed with water followed by brine, dried (Na₂SO₄), and concentrated. The crude mixture was purified by chromatography on SiO₂ (100% hexane to 30-50% EtOAc/hexanes) to obtain 20 (710 mg, 67%) as a white solid. ¹H NMR (400 MHz, CDCl₃) δ 7.45-7.32 (m, 2H), 7.02-6.90 (m, 2H), 3.56 (dd, J=32.3, 11.1 Hz, 2H), 3.16-3.03 (m, 2H), 2.51-24.1 (m, 1H), 2.27 (td, J=13.3, 3.8 Hz, 1H), 1.86-1.57 (m, 4H), 1.55-1.31 (m, 2H), 0.55 (s, 3H), 0.26 (s, 3H); ¹⁹F NMR (376 MHz, CDCl₃) δ-118.7.

### Procedure for the Preparation of 7R Freebase

To a solution of 20 (640 mg, 2.6 mmol) in IPA (4 V) at room temperature was added conc. aq. HCL (4 equiv.), and the mixture was heated at 70° C. for 14 h to obtain a complete conversion of the starting material. The mixture was then basified with a solution of 3N NaOH (aq) and extracted with MTBE. The combined organics were washed once with water, dried (Na₂SO₄), and concentrated to obtain crude 7R freebase (430 mg, 93%) as colorless oil, which was carried to the next step without further purification.

### Procedure for the Preparation of 7R HCl

To a solution of crude 7R freebase (430 mg) in MTBE (5 mL) was added a solution of HCl in IPA (1.5 equiv.) drop-wise at room temperature. Formation of a white suspension was observed during the addition of the HCl solution. The resulting white suspension was then allowed to stir at room temperature for 12-14 h. It was then filtered and washed with MTBE (3×3 V) to obtain 7R HCl (420 mg, 84%) as a white solid. ¹H NMR (400 MHz, DMSO) δ 9.82 (s, 1H), 9.34 (s, 1H), 7.53-7.32 (m, 4H), 3.15 (dt, J=13.8, 3.0 Hz, 1H), 2.45-2.27 (m, 2H), 2.16-2.03 (m, 1H), 2.02-1.79 (m, 2H), 1.72-1.48 (m, 2H); ¹³C NMR (101 MHz, DMSO) δ 265, 164.5, 162.0, 131.6, 131.5, 126.9, 126.9, 117.2, 117.0, 70.8, 40.6, 404, 40.2, 40.0, 39.8, 39.6, 39.4, 39.3, 31.8, 27.5, 26.6, 26.4, 26.2, 21.5; ¹⁹F NMR (376 MHz, DMSO) δ-111.0.

### Example 6. Metabolic Stability in Human Liver Microsomes

Disclosed compounds were tested for stability in human liver microsomes (HLM), with the results summarized in Table 1. Disclosed compounds exhibited greater metabolic stability than ketamine in this model.

Drugs. Compounds were tested as the racemates or pure enantiomers, as indicated. Ketamine was commercially obtained.

HLM Stability. Pooled HLM from adult male and female donors (Corning 452117) were used. Microsomal incubations were carried out in multi-well plates. Liver microsomal incubation medium consisted of PBS (100 mM, pH 7.4), MgCl₂ (1 mM), and NADPH (1 nmM), with 0.50 mg of liver microsomal protein per mL. Control incubations were performed by replacing the NADPH-cofactor system with PBS. Test compounds (1 μM, final solvent concentration 1.0%) were incubated with microsomes at 37° C. with constant shaking, Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/mL labetalol as internal standards (IS), followed by shaking for 10 minutes, and then protein sedimentation by centrifugation at 4,000 rpm for 20 minutes at 4° C. Supernatant samples (80 μL) were diluted with water (240 μL) and analyzed for parent compound remaining using a fit-for-purpose liquid chromatography-tandem mass spectrometry (LC-MS/MS) method.

Data Analysis. The elimination constant (kₑₗ), half-life (t_{1/2}) and intrinsic clearance (Clᵢₙₜ) were determined in a plot of ln(AUC) versus time, using linear regression analysis.

TABLE 1
Intrinsic clearance (Clᵢₙₜ) and half-life (t_{1/2}) of ketamine and disclosed
compounds in the presence of HLM.

| Compound | | Clᵢₙₜ | t_{1/2} |
|---|---|---|---|
| Number | Structure | (μL/min/m | (min) |
| racemic ketamine | 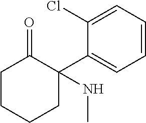 [SMILES: CNC1(c2ccccc2Cl)CCCCC1=O] | 25.5 | 54.4 |
| 1 | 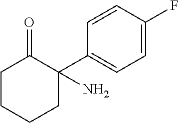 [SMILES: NC1(c2ccc(F)cc2)CCCCC1=O] | <9.6 | >145 |
| 1S | 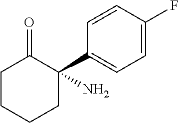 [SMILES: N[C@]1(c2ccc(F)cc2)CCCCC1=O] | <9.6 | >145 |
| 1R | 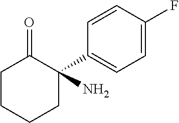 [SMILES: N[C@@]1(c2ccc(F)cc2)CCCCC1=O] | <9.6 | >145 |
| 2 | 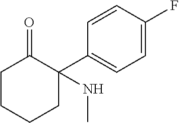 [SMILES: CNC1(c2ccc(F)cc2)CCCCC1=O] | <9.6 | >145 |
| 2S | 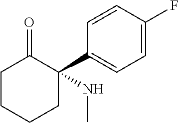 [SMILES: CN[C@]1(c2ccc(F)cc2)CCCCC1=O] | <9.6 | >145 |
| 2R | 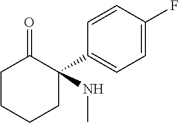 [SMILES: CN[C@@]1(c2ccc(F)cc2)CCCCC1=O] | <9.6 | >145 |
| 3S | 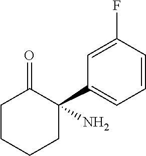 [SMILES: N[C@]1(c2cccc(F)c2)CCCCC1=O] | <9.6 | >145 |
| 3R | 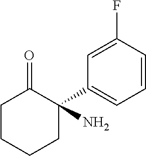 [SMILES: N[C@@]1(c2cccc(F)c2)CCCCC1=O] | <9.6 | >145 |

### Example 7. Metabolic Stability in Mouse Liver Microsomes

Disclosed compounds were tested for stability in mouse liver microsomes (MLM), with the results summarized in Table 2. Disclosed compounds exhibited greater metabolic stability than ketamine in this model.

Drugs. Compounds were tested as the racemates or pure enantiomers, as indicated. Ketamine was commercially obtained.

MLM Stability. Pooled MLM from male CD-1 mice (XenoTech M1000) were used. Microsomal incubations were carried out in multi-well plates. Liver microsomal incubation medium consisted of PBS (100 mM, pH 7.4), MgCl₂ (1 mM), and NADPH (1 mM), with 0.50 mg of liver microsomal protein per mL. Control incubations were performed by replacing the NADPH-cofactor system with PBS. Test compounds (1 μM, final solvent concentration 1.0%) were incubated with microsomes at 37° C. with constant shaking. Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/mL labetalol as internal standards (IS), followed by shaking for 10 minutes, and then protein sedimentation by centrifugation at 4,000 rpm for 20 minutes at 4° C. Supernatant samples (80 μL) were diluted with water (240 μL) and analyzed for parent compound remaining using a fit-for-purpose liquid chromatography-tandem mass spectrometry (LC-MS/MS) method.

Data Analysis. The elimination constant (kₑₗ), half-life (t_{1/2}) and intrinsic clearance (Clᵢₙₜ) were determined in a plot of ln(AUC) versus tire, using linear regression analysis.

TABLE 2
Intrinsic clearance (Clᵢₙₜ) and half-life (t_{1/2}) of ketamine and
disclosed compounds in the presence of MLM.

| Compound | | Clᵢₙₜ | t_{1/2} |
|---|---|---|---|
| Number | Structure | (μL/min/mg) | (min) |
| racemic ketamine | 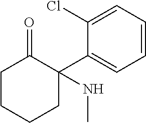 [SMILES: CNC1(c2ccccc2Cl)CCCCC1=O] | 90.1 | 15.4 |
| 1 | 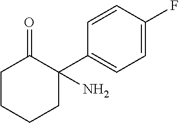 [SMILES: NC1(c2ccc(F)cc2)CCCCC1=O] | <9.6 | >145 |
| 1S | 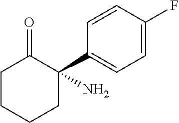 [SMILES: N[C@]1(c2ccc(F)cc2)CCCCC1=O] | <9.6 | >145 |
| 1R | 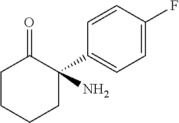 [SMILES: N[C@@]1(c2ccc(F)cc2)CCCCC1=O] | <9.6 | >145 |
| 2 | 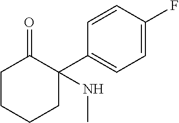 [SMILES: CNC1(c2ccc(F)cc2)CCCCC1=O] | 13.7 | 100.9 |
| 2S | 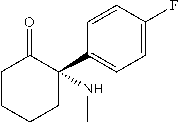 [SMILES: CN[C@]1(c2ccc(F)cc2)CCCCC1=O] | 12.9 | 107.2 |
| 2R | 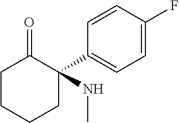 [SMILES: CN[C@@]1(c2ccc(F)cc2)CCCCC1=O] | 10.9 | 127.0 |
| 3S | 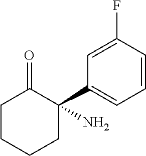 [SMILES: N[C@]1(c2cccc(F)c2)CCCCC1=O] | 11.3 | 122.8 |
| 3R | 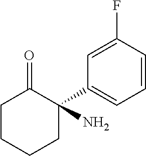 [SMILES: N[C@@]1(c2cccc(F)c2)CCCCC1=O] | 11.6 | 120.0 |

### Example 8. Metabolic Stability in Rat Liver Microsomes

Disclosed compounds were tested for stability in rat liver microsomes (RLM), with the results summarized in Table 3. Disclosed compounds exhibited greater metabolic stability than ketamine in this model. Further, compounds 1, 2, and 3 exhibited much greater stability than their analogs where the fluorine was replaced by a methyl group (compounds 10, 11, and 12, respectively).

Drugs. Compounds were tested as the racemates or pure enantiomers, as indicated. Ketamine was commercially obtained.

RLM Stability. Pooled RLM from male Sprague Dawley rats (XenoTech R1000) were used. Microsomal incubations were carried out in multi-well plates. Liver microsomal incubation medium consisted of PBS (100 mM, pH 7.4), MgCl₂ (1 mM), and NADPH (1 mM), with 0.50 mg of liver microsomal protein per mL. Control incubations were performed by replacing the NADPH-cofactor system with PBS. Test compounds (1 μM, final solvent concentration 1.0%) were incubated with microsomes at 37° C. with constant shaking. Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/mL labetalol as internal standards (IS), followed by shaking for 10 minutes, and then protein sedimentation by centrifugation at 4,000 rpm for 20 minutes at 4° C. Supernatant samples (80 μL) were diluted with water (240 μL) and analyzed for parent compound remaining using a fit-for-purpose liquid chromatography-tandem mass spectrometry (LC-MS/MS) method.

Data Analysis. The elimination constant (kₑₗ), half-life (t_{1/2}) and intrinsic clearance (Clᵢₙₜ) were determined in a plot of ln(AUC) versus time, using linear regression analysis.

TABLE 3
Intrinsic clearance (Clᵢₙₜ) and half-life (t_{1/2}) of ketamine and disclosed compounds in the
presence of RLM.

| Compound | | | |
|---|---|---|---|
| Number | | | |
| (rac = | | Clᵢₙₜ | t_{1/2} |
| racemic) | Structure | (μL/min/mg) | (min) |
| rac-ketamine | 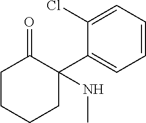 [SMILES: CNC1(c2ccccc2Cl)CCCCC1=O] | 294 | 5.6 |
| 1 | 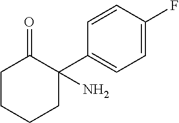 [SMILES: NC1(c2ccc(F)cc2)CCCCC1=O] | <9.6 | >145 |
| 1S | 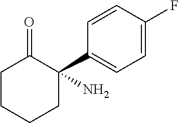 [SMILES: N[C@]1(c2ccc(F)cc2)CCCCC1=O] | <9.6 | >145 |
| 1R | 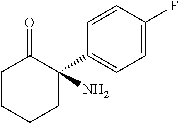 [SMILES: N[C@@]1(c2ccc(F)cc2)CCCCC1=O] | <9.6 | >145 |
| 2 | 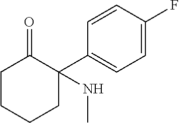 [SMILES: CNC1(c2ccc(F)cc2)CCCCC1=O] | 19.6 | 70.6 |
| 2S | 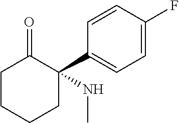 [SMILES: CN[C@]1(c2ccc(F)cc2)CCCCC1=O] | 22.8 | 60.8 |
| 2R | 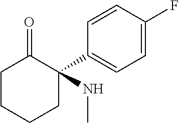 [SMILES: CN[C@@]1(c2ccc(F)cc2)CCCCC1=O] | 17.8 | 78.1 |
| 3S | 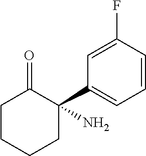 [SMILES: N[C@]1(c2cccc(F)c2)CCCCC1=O] | 14.6 | 94.6 |
| 3R | 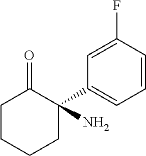 [SMILES: N[C@@]1(c2cccc(F)c2)CCCCC1=O] | <9.6 | >145 |
| 10 | 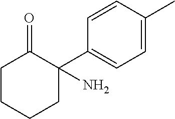 | 379.8 | 3.6 |
| 11 | 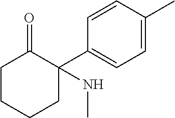 [SMILES: CNC1(c2ccc(C)cc2)CCCCC1=O] | 416.7 | 3.3 |
| 12 | 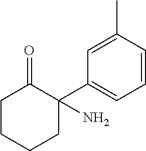 | 191.7 | 7.2 |

### Example 9. Metabolic Stability in Dog Liver Microsomes

Disclosed compounds were tested for stability in dog liver microsomes (DLM), with the results summarized in Table 4. Disclosed compounds were moderately to highly stable in this model. Compound 2R was substantially more stable in DLM than its enantiomer 2S.

Drugs. Compounds were tested as the racemates or pure enantiomers, as indicated. Ketamine was commercially obtained.

DLM Stability. Pooled DLM from male beagle dogs (XenoTech D1000) were used. Microsomal incubations were carried out in multi-well plates. Liver microsomal incubation medium consisted of PBS (100 mM, pH 7.4), MgCl₂ (1 mM), and NADPH (1 mM), with 0.50 mg of liver microsomal protein per mL. Control incubations were performed by replacing the NADPH-cofactor system with PBS. Test compounds (1 μM, final solvent concentration 1.0%) were incubated with microsomes at 37° C. with constant shaking. Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/mL labetalol as internal standards (TS), followed by shaking for 10 minutes, and then protein sedimentation by centrifugation at 4,000 rpm for 20 minutes at 4° C. Supernatant samples (80 μL) were diluted with water (240 μL) and analyzed for parent compound remaining using a fit-for-purpose liquid chromatography-tandem mass spectrometry (LC-MS/MS) method.

Data Analysis. The elimination constant (kₑₗ), half-life (t_{1/2}) and intrinsic clearance (Clᵢₙₜ) were determined in a plot of ln(AUC) versus time, using linear regression analysis.

TABLE 4
Intrinsic clearance (Clᵢₙₜ) and half-life (t_{1/2}) of disclosed compounds in the presence of
DLM.

| Compound | | | |
|---|---|---|---|
| Number | | | |
| (rac = | | | t_{1/2} |
| racemic) | Structure | Clᵢₙₜ (μL/min/mg) | (min) |
| rac-ketamine | 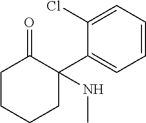 [SMILES: CNC1(c2ccccc2Cl)CCCCC1=O] | 589 | 2.4 |
| 1S | 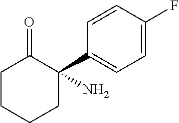 [SMILES: N[C@]1(c2ccc(F)cc2)CCCCC1=O] | <9.6 | >145 |
| 2S | 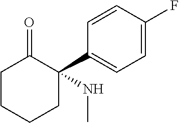 [SMILES: CN[C@]1(c2ccc(F)cc2)CCCCC1=O] | 76.0 | 18.2 |
| 2R | 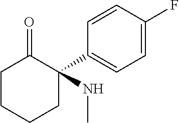 [SMILES: CN[C@@]1(c2ccc(F)cc2)CCCCC1=O] | 35.8 | 38.7 |
| 3S | 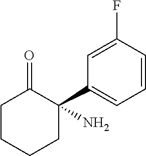 [SMILES: N[C@]1(c2cccc(F)c2)CCCCC1=O] | 26.8 | 51.7 |
| 3R | 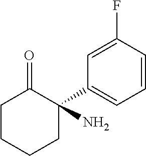 [SMILES: N[C@@]1(c2cccc(F)c2)CCCCC1=O] | 34.6 | 40.0 |

### Example 10. Metabolic Stability in Monkey Liver Microsomes

Disclosed compounds were tested for stability in cynomolgus monkey liv er microsomes (CLM), with the results summarized in Table 5, Disclosed compounds were moderately to highly stable in this model. Compound 2R was substantially more stable in CLM than its enantiomer 2S.

Drugs. Compounds were tested as the racemates or pure enantiomers, as indicated. Ketamine was commercially obtained.

CLM Stability. Pooled CLM from male cynomolgus monkeys (Corning 452413) were used. Microsomal incubations were carried out in multi-well plates. Liver microsomal incubation medium consisted of PBS (100 mM, pH 7.4), MgCl₂ (1 mM), and NADPH (1 mM), with 0.50 mg of liver microsomal protein per mL. Control incubations were performed by replacing the NADPH-cofactor system with PBS. Test compounds (1 μM, final solvent concentration 1.0%) were incubated with microsomes at 37° C. with constant shaking. Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/mL labetalol as internal standards (IS), followed by shaking for 10 minutes, and then protein sedimentation by centrifugation at 4,000 rpm for 20 minutes at 4° C. Supernatant samples (80 μL) were diluted with water (240 μL) and analyzed for parent compound remaining using a fit-for-purpose liquid chromatography-tandem mass spectrometry (LC-MS/MS) method.

Data Analysis. The elimination constant (kₑₗ), half-life (t_{1/2}) and intrinsic clearance (Clᵢₙₜ) were determined in a plot of ln(AUC) versus time, using linear regression analysis.

TABLE 5
Intrinsic clearance (Clᵢₙₜ) and half-life (t_{1/2}) of disclosed compounds in the presence of
CLM.

| Compound | | Clᵢₙₜ | t_{1/2} |
|---|---|---|---|
| Number | Structure | (μL/min/mg) | (min) |
| 1S | 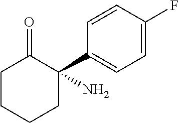 [SMILES: N[C@]1(c2ccc(F)cc2)CCCCC1=O] | <9.6 | >145 |
| 2S | 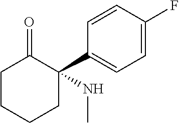 [SMILES: CN[C@]1(c2ccc(F)cc2)CCCCC1=O] | 36.1 | 38.3 |
| 2R | 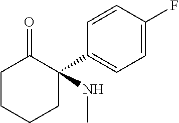 [SMILES: CN[C@@]1(c2ccc(F)cc2)CCCCC1=O] | 22.2 | 62.3 |
| 3S | 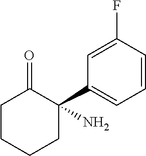 [SMILES: N[C@]1(c2cccc(F)c2)CCCCC1=O] | 16.6 | 83.7 |
| 3R | 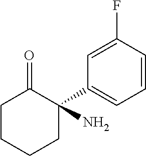 [SMILES: N[C@@]1(c2cccc(F)c2)CCCCC1=O] | 12.5 | 110.9 |

### Example 11. Metabolic Stability in Minipig Liver Microsomes

Disclosed compounds were tested for stability in Gottingen minipig liver microsomes (MPLM), with the results summarized in Table 6. Disclosed compounds were moderately to highly stable in this model.

Drugs. Compounds were tested as the racemates or pure enantiomers, as indicated. Ketamine was commercially obtained.

MPLM Stability. Pooled MPLM from Gottingen minipigs (Xenotech Z6000) were used. Microsomal incubations were carried out in multi-well plates. Liver microsomal incubation medium consisted of PBS (100 mM pH 7.4), MgCl₂ (1 mM), and NADPH (1 mM), with 0.50 mg of liver microsomal protein per mL. Control incubations were performed by replacing the NADPH-cofactor system with PBS. Test compounds (1 μM, final solvent concentration 1.0%) were incubated with microsomes at 37° C. with constant shaking. Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/mL labetalol as internal standards (IS), followed by shaking for 10 minutes, and then protein sedimentation by centrifugation at 4,000 rpm for 20 minutes at 4° C. Supernatant samples (80 μL) were diluted with water (240 μL) and analyzed for parent compound remaining using a fit-for-purpose liquid chromatography-tandem mass spectrometry (LC-MS/MS) method.

Data Analysis. The elimination constant (kₑₗ), half-life (t_{1/2}) and intrinsic clearance (Clᵢₙₜ) were determined in a plot of ln(AUC) versus time, using linear regression analysis.

TABLE 6
Intrinsic clearance (Clᵢₙₜ) and half-life (t_{1/2}) of disclosed compounds in the presence of
MPLM.

| Compound | | | t_{1/2} |
|---|---|---|---|
| Number | Structure | Clint (μL/min/mg) | (min) |
| rac-ketamine | 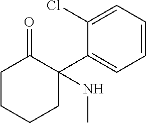 [SMILES: CNC1(c2ccccc2Cl)CCCCC1=O] | 218 | 6.4 |
| 1S | 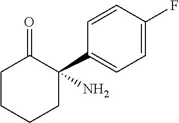 [SMILES: N[C@]1(c2ccc(F)cc2)CCCCC1=O] | <9.6 | >145 |
| 2S | 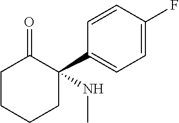 [SMILES: CN[C@]1(c2ccc(F)cc2)CCCCC1=O] | 44.9 | 30.9 |
| 2R | 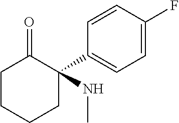 [SMILES: CN[C@@]1(c2ccc(F)cc2)CCCCC1=O] | 36.5 | 38 |
| 3S | 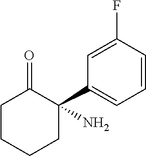 [SMILES: N[C@]1(c2cccc(F)c2)CCCCC1=O] | <9.6 | >145 |

### Example 12. Oral Bioavailability in Mice

In mice, disclosed compounds demonstrated improved absolute oral bioavailability (F), longer half-life (t_{1/2}), higher maximal concentrations (Cₘₐₓ) (when corrected for dose), and higher absolute exposure as quantified by area under the curve (AUC) (when corrected for dose), compared to ketamine in both plasma (Table 7) and brain (Table 8). Compound 2R exhibited substantially higher brain exposure after oral administration compared to its enantiomer 2S.

### Method A:

Animals. Male CD-1 mice were used in these studies. Animals were randomly assigned to treatment groups and were fasted for 4 h before dosing.

Drugs. Test compounds were dissolved in normal saline and administered intravenously (iv) or orally (po) at a dose of 10 mg/kg (calculated based on freebase) and at a volume of 5 mL/kg body weight. Compounds were tested as the racemates or pure enantiomers, as indicated.

Sample Collection and Bioanalysis. Blood samples were collected under 2,2,2-tribromoethanol anesthesia (150 mg/kg, ip) from the orbital sinus at 0.083, 0.25, 0.5, 1, 2, 4, 8 and 24 h (4 animals per time point) into microcontainers containing K₂EDTA. Immediately after collection of blood, mice were euthanized by cervical dislocation and brain samples were collected at the same time points. All samples were immediately processed, flash-frozen, and stored at −70° C. until subsequent analysis. Plasma samples were separated by centrifugation of is whole blood and aliquots (50 μL) were mixed with 200 μL of internal standard solution (400 ng/mL in 1:1 v/v CH₃CN:MeOH). After mixing by pipetting and centrifuging for 4 min at 6,000 rpm, 0.5 μL of each supernatant was analyzed for drug using a fit-for-purpose liquid chromatography-tandem mass spectrometry (LC-MS/MS) method, with authentic samples of each analyte used for calibration and identification. Brain samples (weight 100 mg±1 mg) were dispersed in 500 μL of internal standard solution (400 ng/mL in 4:1 v/v MeOH:water) using zirconium oxide beads (115 mg±5 mg) in The Bullet Blender® homogenizer for 30 s at speed 8. After homogenization, the samples were centrifuged for 4 min at 14,000 rpm and 0.5 μL of each supernatant was analyzed for drug using a fit-for-purpose LC-MS/MS method, with authentic samples of each analyte used for calibration and identification.

Data Analysis. The drug concentrations of samples below the lower limit of quantitation (LLOQ) were designated as zero. Pharmacokinetic data analysis was performed using noncompartmental, bolus injection or extravascular input analysis models in WinNonlin 5.2 (PharSight). Data points below LLOQ were presented as missing to improve validity of tin calculations.

### Method B:

Animals. Male C57BL/6 mice were used in these studies. Animals were randomly assigned to treatment groups and were fasted for 4 h before dosing.

Drugs. Test compounds were dissolved in a vehicle consisting of normal saline (for compounds used as the HCl salt) or normal saline slightly acidified with aq. HCl (for freebase compounds). They were then administered intravenously (iv) or orally (po) at a dose of 1 or 10 mg/kg (calculated based on freebase), as indicated, and at a volume of 5 mL/kg body weight. Compounds were tested as the racemates or pure enantiomers, as indicated.

Sample Collection and Bioanalysis. Blood samples (approximately 60 μL) were collected under light isoflurane anesthesia (Surgivet®) from the retro orbital plexus at 0.08, 0.25, 0.5, 1, 2, 4, 8, and 24 h (4 animals per time point). Immediately after blood collection, plasma was harvested by centrifugation at 4,000 rpm for 10 min at 4° C. and samples were stored at −70±10° C. until bioanalysis. Following blood collection, animals were immediately sacrificed, the abdominal vena-cava was cut open, and the whole body was perfused from the heart using 10 mL of normal saline, and brain samples were collected from all animals. After isolation, brain samples were rinsed three times in ice-cold normal saline (for 5-10 seconds/rinse using ˜5-10 mL, normal saline in disposable petri dish for each rinse) and dried on blotting paper. Brain samples were homogenized using ice-cold phosphate-buffered saline (pH 7.4). Total homogenate volume was three times the tissue weight. All homogenates were stored at −70±10° C. until bioanalysis. For bioanalysis, 25 μL aliquots of plasma/brain study samples or spiked plasma/brain calibration standards were added to individual pre-labeled micro-centrifuge tubes followed by 100 μL of an internal standard solution (glipizide, 500 ng/mL in acetonitrile) except for blanks, where 100 μL of acetonitrile was added. Samples were vortexed for 5 minutes and then centrifuged for 10 minutes at 4,000 rpm at 4° C. Following centrifugation, 100 μL of each clear supernatant was transferred to a 96 well plate and analyzed with a fit-for-purpose LC-MS/MS method, with authentic samples of each analyte used for calibration and identification.

Data Analysis. Pharmacokinetic parameters were estimated using the non-compartmental analysis tool of Phoenix® WinNonlin software (Ver 8.0).

TABLE 7
Selected pharmacokinetic parameters of ketamine and disclosed compounds in plasma of mice.

| | | | | Cₘₐₓ | | | t_{1/2} | t_{1/2} | |
|---|---|---|---|---|---|---|---|---|---|
| Compound | | | Dose | (po) | AUC_{0-inf} (iv) | AUC_{0-inf} (po) | (iv) | (po) | F |
| Number | Structure | Method | (mg/kg) | (ng/mL) | (ng*min/mL)* | (ng*min/mL)* | (min) | (min) | % |
| racemic ketamine | 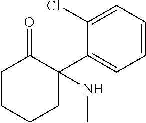 [SMILES: CNC1(c2ccccc2Cl)CCCCC1=O] | A | 10 | 253 | 38,000 | 5,810 | 8.46 | 11.5 | 15 |
| 1S | 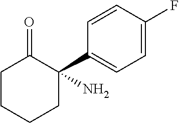 [SMILES: N[C@]1(c2ccc(F)cc2)CCCCC1=O] | B | 1 | 394 | 30,067 | 28,215 | 86.4 | 82.2 | 94 |
| 2S | 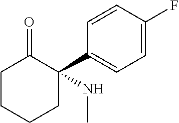 [SMILES: CN[C@]1(c2ccc(F)cc2)CCCCC1=O] | B | 1 | 51.7 | 7,148 | 2,796 | 54.6 | 50.4 | 39 |
| 2R | 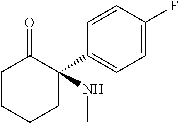 [SMILES: CN[C@@]1(c2ccc(F)cc2)CCCCC1=O] | B | 1 | 53.6 | 5,779 | 2,328 | 45.0 | 29.4 | 40 |
| 3S | 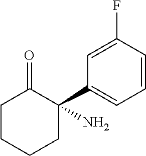 [SMILES: N[C@]1(c2cccc(F)c2)CCCCC1=O] | B | 1 | 640 | 36,509 | 32,511 | 101 | 29.4 | 89 |

*For parameters determined by method B, AUC values represent AUC₀₋ₗₐₛₜ and calculated F is based on these values rather than on AUC_{0-inf}.

TABLE 8
Selected pharmacokinetic parameters of ketamine and disclosed compounds in brains of mice.

| Compound | | | | | | | | | |
|---|---|---|---|---|---|---|---|---|---|
| Number | | | | Cₘₐₓ | | AUC_{0-inf} | t_{1/2} | t_{1/2} | |
| (rac | | | Dose | (po) | AUC_{0-inf} (iv) | (po) | (iv) | (po) | F |
| racemic) | Structure | Method | (mg/kg) | (ng/g) | (ng*min/g)* | (ng*min/g)* | (min) | (min) | (%) ** |
| rac- ketamine | 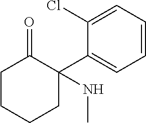 [SMILES: CNC1(c2ccccc2Cl)CCCCC1=O] | A | 10 | 521 | 97,000 | 6,030 | 8.66 | 12.2 | 6.2 |
| 1S | 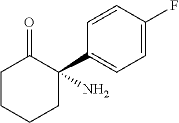 [SMILES: N[C@]1(c2ccc(F)cc2)CCCCC1=O] | B | 1 | 82.2 | 14,776 | 6,557 | 36.6 | NC | 44 |
| 2S | 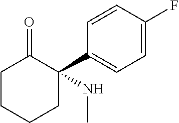 [SMILES: CN[C@]1(c2ccc(F)cc2)CCCCC1=O] | B | 1 | 80.4 | 21,532 | 4,264 | 21.6 | 42.0 | 20 |
| 2R | 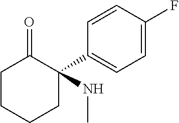 [SMILES: CN[C@@]1(c2ccc(F)cc2)CCCCC1=O] | B | 1 | 177 | 17,791 | 5,890 | 23.4 | 36.0 | 33 |
| 3S | 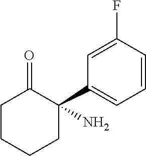 [SMILES: N[C@]1(c2cccc(F)c2)CCCCC1=O] | B | 1 | 112 | 18,797 | 7,418 | 26.4 | 40.8 | 39 |

*For parameters determined by method B, AUC values represent AUC₀₋ₗₐₛₜ and calculated F is based on these values rather than on AUC_{0-inf}.
**Calculated based on brain exposure.

### Example 13. Oral Bioavailability in Rats

In rats, disclosed compounds demonstrated improved absolute oral bioavailability (F), longer half-life (t_{1/2}), higher maximal concentrations (Cₘₐₓ) (when corrected for dose), and higher absolute exposure as quantified by area under the curve (AUC) (when corrected for dose), compared to ketamine in both plasma (Table 9) and brain (Table 10). Compound 2R exhibited substantially higher brain exposure after oral administration compared to its enantiomer 2S.

### Method A:

Animals. Male Sprague Dawley rats were used in these studies. Animals were randomly assigned to treatment groups and were fasted for 4 h before dosing.

Drugs. Test compounds were dissolved in normal saline and administered intravenously (iv) or orally (po) at a dose of 10 mg/kg (calculated based on freebase) and at a volume of 5 mL/kg body weight. Compounds were tested as the racemates or pure enantiomers, as indicated.

Sample Collection and Bioanalysis. Blood samples were collected under 2,2,2-tribromoethanol anesthesia (150 mg/kg, ip) from the orbital sinus at 0.083, 0.25, 0.5, 1, 2, 4, 8 and 24 h (4 animals per time point) into microcontainers containing K₂EDTA. Immediately after collection of blood, rats were euthanized by cervical. All samples were immediately processed, flash-frozen, and stored at −70° C. until subsequent analysis. Plasma samples were separated by centrifugation of whole blood and aliquots (50 μL) were mixed with 200 μL of internal standard solution (400 ng/mL in 1:1 v/v CH₃CN:MeOH). After mixing by pipetting and centrifuging for 4 min at 6,000 rpm, 0.5 μL of each supernatant was analyzed for drug using a fit-for-purpose liquid chromatography-tandem mass spectrometry (LC-MS/MS) method, with authentic samples of each analyte used for calibration and identification.

Data Analysis. The drug concentrations of samples below the lower limit of quantitation (LLOQ) were designated as zero. Pharmacokinetic data analysis was performed using noncompartmental, bolus injection or extravascular input analysis models in WinNonlin 5.2 (PharSight). Data points below LLOQ were presented as missing to improve validity of t_{1/2} calculations.

### Method B:

Animals. Male Sprague Dawley rats were used in these studies. Animals were randomly assigned to treatment groups and were fasted for 4 h before dosing.

Drugs. Test compounds were dissolved in a vehicle consisting of normal saline (for compounds used as the HCl salt) or normal saline slightly acidified with aq. HCl (for freebase compounds). They were then administered intravenously (iv) or orally (po) at a dose of 1 or 10 ng/kg (calculated based on freebase), as indicated, and at a volume of 5 mL/kg body weight. Compounds were tested as the racemates or pure enantiomers, as indicated.

Sample Collection and Bioanalysis. Blood samples (approximately 60 μL) were collected under light isoflurane anesthesia (Surgivet®) from the retro orbital plexus at 0.08, 0.25, 0.5, 1, 2, 4, 8, and 24 h (4 animals per time point). Immediately after blood collection, plasma was harvested by centrifugation at 4,000 rpm for 10 min at 4° C. and samples were stored at −70±10° C. until bioanalysis. Following blood collection, animals were immediately sacrificed, the abdominal vena-cava was cut open, and the whole body was perfused from the heart using 10 mL of normal saline, and brain samples were collected from all animals. After isolation, brain samples were rinsed three times in ice-cold normal saline (for 5-10 seconds/rinse using ˜-5-10 mL normal saline in disposable petri dish for each rinse) and dried on blotting paper. Brain samples were homogenized using ice-cold phosphate-buffered saline (pH 7.4). Total homogenate volume was three times the tissue weight. All homogenates were stored at −70±10° C. until bioanalysis. For bioanalysis, 25 μL aliquots of plasma/brain study samples or spiked plasma/brain calibration standards were added to individual pre-labeled micro-centrifuge tubes followed by 100 μL of an internal standard solution (glipizide, 500 ng/mL in acetonitrile) except for blanks, where 100 μL of acetonitrile was added. Samples were vortexed for 5 minutes and then centrifuged for 10 minutes at 4,000 rpm at 4° C. Following centrifugation, 100 μL of each clear supernatant was transferred to a 96 well plate and analyzed with a fit-for-purpose LC-MS/MS method, with authentic samples of each analyte used for calibration and identification.

Data Analysis. Pharmacokinetic parameters were estimated using the non-compartmental analysis tool of Phoenix® WinNonlin software (Ver 8.0).

TABLE 9
Selected pharmacokinetic parameters of ketamine and disclosed compounds in plasma of rats.

| | | | | Cₘₐₓ | | | t_{1/2} | t_{1/2} | |
|---|---|---|---|---|---|---|---|---|---|
| Compound | | | Dose | (po) | AUC_{0-inf} (iv) | AUC_{0-inf} (po) | (iv) | (po) | F |
| Number | Structure | Method | (mg/kg) | (ng/mL) | (ng*min/mL.)* | (ng*min/mL)* | (min) | (min) | (%) |
| racemic ketamine | 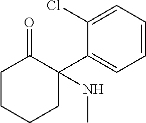 [SMILES: CNC1(c2ccccc2Cl)CCCCC1=O] | A | 10 | 190 | 81000 | 7500 | 41.58 | 33.72 | 9.07 |
| 1S | 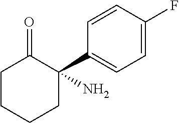 [SMILES: N[C@]1(c2ccc(F)cc2)CCCCC1=O] | B | 1 | 233.5 | 48290.4 | 68401.8 | 154.8 | 232.8 | >100 |
| 2S | 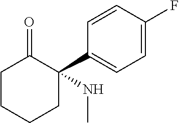 [SMILES: CN[C@]1(c2ccc(F)cc2)CCCCC1=O] | B | 1 | 32.31 | 11132.4 | 2266.8 | 42 | 87.6 | 20 |
| 2R | 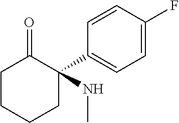 [SMILES: CN[C@@]1(c2ccc(F)cc2)CCCCC1=O] | B | 1 | 17.73 | 9780 | 3017.4 | 166.2 | 94.8 | 31 |

*For parameters determined by method B, AUC values represent AUC₀₋ₗₐₛₜ and calculated F is based on these values rather than on AUC_{0-inf}.

TABLE 10
Selected pharmacokinetic parameters of ketamine and disclosed compounds in brains of rats.

| Compound | | | | | | | | | |
|---|---|---|---|---|---|---|---|---|---|
| Number | | | | Cₘₐₓ | | AUC_{0-inf} | t_{1/2} | t_{1/2} | |
| (rac | | | Dose | (po) | AUC_{0-inf} (iv) | (po) | (iv) | (po) | F |
| racemic) | Structure | Method | (mg/kg) | (ng/g) | (ng*min/g)* | (ng*min/g)* | (min) | (min) | (%) ** |
| 1S | 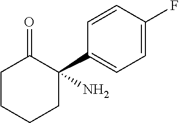 [SMILES: N[C@]1(c2ccc(F)cc2)CCCCC1=O] | B | 1 | 166.73 | 126462 | 79272.6 | 245.4 | 426.6 | 62.7 |
| 2S | 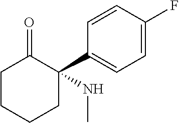 [SMILES: CN[C@]1(c2ccc(F)cc2)CCCCC1=O] | B | 1 | 47.58 | 68000.4 | 3067.2 | 42 | NC | 4.5 |
| 2R | 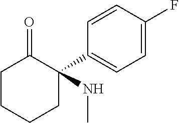 [SMILES: CN[C@@]1(c2ccc(F)cc2)CCCCC1=O] | B | 1 | 42.75 | 27152.4 | 4915.8 | 3.8 | 59.4 | 18.1 |

*For parameters determined by method B, AUC values represent AUC₀₋ₗₐₛₜ and calculated F is based on these values rather than on AUC_{0-inf}.
**Calculated based on brain exposure.
NC = not calculated

### Example 14. Oral Bioavailability in Minipigs

In minipigs, compound 2R showed good oral bioavailability (Table 11).

### Method:

Animals. Male Bama minipigs were used in these studies. Animals were randomly assigned to treatment groups and were fasted overnight before dosing.

Drugs. Compound 2R was dissolved in a vehicle consisting of normal saline. It was then administered intravenously (iv) or orally (po) at a dose of 1 mg/kg (calculated based on freebase) and at a volume of 2 mL/kg body weight (n=3 per dosing route).

Sample Collection and Bioanalysis. Blood samples (approximately 500 μL) were collected under manual restraint from the cephalic vein at 0.08, 0.25, 0.5, 1, 2, 4, 8, and 24 h (4 animals per time point) into K2EDTA tubes and placed on wet ice. Immediately after blood collection, plasma was harvested by centrifugation at 3,000 g for 5 min at 4° C. within 15 minutes of collection and subsequently stored at −70±10° C. until bioanalysis. For bioanalysis, for diluted plasma samples, an aliquot of 2 μL sample was diluted with 18 μL blank matrix and the dilution factor was 10. For non-diluted samples, an aliquot of 20 μL sample was added with 300 μL internal standard (diclofenac, 60 ng/mL) in acetonitrile. The mixture was vortexed for 10 minutes and centrifuged at 5,800 rpm for 10 minutes. 90 μL of supernatant was transferred to a 96 well plate and analyzed with a fit-for-purpose LC-MS/MS method, with authentic samples of each analyte used for calibration and identification.

Data Analysis. Pharmacokinetic parameters were estimated using the non-compartmental analysis tool of Phoenix® WinNonlin software (Ver 8.2).

TABLE 11
Selected pharmacokinetic parameters of compound 2R in plasma of minipigs.

| Compound | | Dose | AUC_{0-inf} (iv) | AUC_{0-inf} (po) | F |
|---|---|---|---|---|---|
| Number | Structure | (mg/kg) | (ng*min/mL) | (ng*min/mL) | (%) |
| 2R | 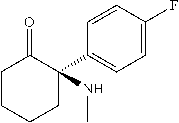 [SMILES: CN[C@@]1(c2ccc(F)cc2)CCCCC1=O] | 1 | 33960 | 14520 | 42.7 |

### Example 15. Oral Bioavailability in Monkeys

In monkeys, compound 2R exhibited moderate oral bioavailability (Table 12).

### Method:

Animals. Male Cynomolgus monkeys were used in these studies. Animals were randomly assigned to treatment groups and were fasted overnight before dosing.

Drugs. Compound 2R was dissolved in a vehicle consisting of normal saline. It was then administered intravenously (iv) or orally (po) at a dose of 1 mg/kg (calculated based on freebase) and at a volume of 2 mL/kg body weight (n=3 per dosing route).

Sample Collection and Bioanalysis. Blood samples (approximately 500 μL) were collected under manual restraint from the cephalic vein at 0.08, 0.25, 0.5, 1, 2, 4, 8, and 24 h (4 animals per time point) into K2EDTA tubes and placed on wet ice. Immediately after blood collection, plasma was harvested by centrifugation at 3,000 g for 5 min at 4° C. within 15 minutes of collection and subsequently stored at −70±10° C. until bioanalysis. For bioanalysis, for diluted plasma samples, an aliquot of 2 μL sample was diluted with 18 μL blank matrix and the dilution factor was 10. For non-diluted samples, an aliquot of 20 μL sample was added with 300 μL internal standard (diclofenac, 60 ng/mL) in acetonitrile. The mixture was vortexed for 10 minutes and centrifuged at 5,800 rpm for 10 minutes. 90 μL of supernatant was transferred to a 96 well plate and analyzed with a fit-for-purpose LC-MS/MS method, with authentic samples of each analyte used for calibration and identification.

Data Analysis. Pharmacokinetic parameters were estimated using the non-compartmental analysis tool of Phoenix® a WinNonlin software (Ver 8.2).

TABLE 12
Selected pharmacokinetic parameters of compound 2R in plasma of monkeys.

| Compound | | Dose | AUC_{0-inf} (iv) | AUC_{0-inf} (po) | F |
|---|---|---|---|---|---|
| Number | Structure | (mg/kg) | (ng*min/mL) | (ng*min/mL) | (%) |
| 2R | 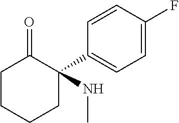 [SMILES: CN[C@@]1(c2ccc(F)cc2)CCCCC1=O] | 1 | 57840 | 15540 | 27 |

### Example 16. NMDA Receptor Binding

The binding affinities of disclosed compounds at the MK-801 binding site of the N-methyl-D-aspartate receptor (NMDAR) were determined in radioligand binding experiments (Table 13). The value shown for racemic ketamine (rac-ketamine) is draw-n from the literature (Ebert et al. 1997). The compounds of the present disclosure exhibited affinity similar to (R)-ketamine and in the ideal range of 1-5 μM for achieving useful therapeutic effects with limited dissociative side effects. Among the compounds tested, 2R had the weakest binding affinity for NMDAR, and was also ˜3-fold less potent than its enantiomer 2S, suggesting that 2R may have a lower potential for dissociative side effects than 2S and the other compounds.

TABLE 13
Binding affinity at the MK-801 site of NMDAR.

| Compound | NMDAR Kᵢ (95% CI) (μM) | |
|---|---|---|
| rac-ketamine | 0.53 ± 0.078* (SEM) | |
| (R)-ketamine | 2.2 | (1.6-2.9) |
| (S)-ketamine | 0.70 | (0.3-1.4) |
| 1S | 1.7 | (1.3-2.3) |
| 2S | 1.2 | (0.79-1.8) |
| 2R | 3.3 | (2.2-4.8) |

| | |
|---|---|
| 3S | 1.7 |

*Ebert et al. 1997

Radioligand Binding. Affinity of the test compounds for NMDAR was determined in radioligand binding experiments with [³H]MK-801 by Eurofins Panlabs, Inc., using methods adapted from the literature (Javitt el al. 1987; Reynolds et al. 1989) and under the conditions described in Table 14.

TABLE 14
NMDAR radioligand binding experimental parameters.

| Receptor Source | Wistar rat brain (minus cerebellum) | |
|---|---|---|
| Vehicle | 1.0% DMSO | |
| Incubation Time | 3 | h |
| Incubation Temperature | 25° | C. |
| Incubation Buffer | 5 mM Tris-HCl, pH 7.4 | |
| Ligand | 5.0 nM [³H]MK-801 | |
| Non-Specific Ligand | 10.0 μM (+)-MK-801 | |
| Specific Binding | 90%* | |
| K_{d} | 12.0 | nM* |

| | |
|---|---|
| Bₘₐₓ | 1.30 pmol/mg protein* |

*historical values

### Example 17. Functional Activity at SERT

The ability of disclosed compounds to inhibit uptake of monoamines by the serotonin transporter (SERT) was measured using a fluorescent substrate uptake assay in transfected cells (Table 15). The compounds varied in their ability to inhibit SERT, with certain compounds (e.g., 2R) demonstrating substantial inhibitor, activity in the micromolar range, while others (e.g., 3S) were inactive at 10 μM. Compound 2R was also more active than its enantiomer 2S. Considering that inhibitors of SERT are well known to have antidepressant and anxiolytic effects and are among the most commonly prescribed drugs for mood disorders (e.g. fluoxetine, sertraline, etc.), blockade of SERT by certain compounds of the present disclosure is expected to synergize with their NMDAR inhibition to increase therapeutic activity for treating depression and related disorders. Indeed, such synergy between these two mechanisms of action has been demonstrated in animal models (Ates-Alagoz and Adejare 2013). Further, the ability to tune the ratio between SERT and NMDAR is useful to obtain the optimal therapeutic profile depending on the intended clinical indication. For example, compounds with greater selectivity for NMDAR might be preferred treatments for patients who are intolerant of the side effects of SERT inhibitors.

TABLE 15
Uptake inhibition activity at SERT.

| | SERT % Uptake |
|---|---|
| Compound | Inhibition @ 10 μM |
| 1S | 14.9 |
| 1R | 10.4 |
| 25 | 36.4 |
| 2R | 58.8 |
| 3S | −0.21 |
| 3R | 1.75 |

Uptake Inhibition. The ability of test compounds to block monoamine uptake by SERT was determined using the Neurotransmitter Transporter Uptake Assay Kit manufactured by Molecular Devices (Cat #R8173). Briefly, stably transfected cells expressing SERT were grown and plated into 384-well plates at a concentration of 20,000 cells per well. Plates were then incubated for 16-20 h at 37° C. and 5% CO₂. The medium was then aspirated and replaced with 25 μL of assay buffer (20 mM HEPES in HBSS, containing 0.1% BSA) containing the test compounds at the appropriate concentrations. Plates were then centrifuged at 300 rpm for 15 s and then incubated at 37° C. for 30 minutes. At this time, 25 μL of the proprietary fluorescent dye solution was added, the plates were incubated at 37° C. for 60 minutes, and then fluorescence was quantified on a plate reader (excitation wavelength=440 nm, emission wavelength=520 nm). The proprietary dye solution contains a mixture of 1) a fluorescent dye that mimics the endogenous substrate of SERT and is thereby actively transported to the intracellular compartment in the absence of an inhibitor and 2) a masking dye that inhibits the fluorescence of dye 1 in the extracellular compartment. Therefore, the overall fluorescence of the system increases as the fluorescent dye is transported into the cells. In the presence of an inhibitor of SERT, uptake of the dye is reduced, and therefore, the fluorescence is also decreased, allowing this inhibition to be quantified.

### Example 18. SERT Binding Affinity

Disclosed compounds were tested for their binding affinity at the serotonin transporter (SERT) using a competition radioligand binding assay (Eurofins Cerep). Assay conditions are described in Table 16 below. The results are shown in Table 17. Both 2S and 2R showed significant binding to SERT, with Kᵢ values of 12 and 6.2 μM, respectively, but the 2R isomer was ˜2-fold more potent. Since blockade of SERT is an important mechanism for antidepressants, the greater affinity for SERT of 2R compared to 2S is likely to afford the 2R isomer with better antidepressant activity than the 2S isomer. Further, both isomers of 2 were substantially more potent as SERT ligands than the structurally related compound 2-(2-fluorophenyl)-2-(methylanino)cyclohexan-1-one (2-F-DCK), suggesting superior antidepressant activity for either isomer of 2 when compared to 2-F-DCK.

TABLE 16
Conditions for SERT binding assay,

| Receptor Source | Human recombinant (CHO cells) | |
|---|---|---|
| Vehicle | 1.0% DMSO | |
| Incubation Time | 1 | h |
| Incubation Temperature | 25° | C. |
| Incubation Buffer | 5 mM Tris-HCl, pH 7.4 | |
| Ligand | 2.0 nM [³H]imipramine | |
| Non-Specific Ligand | 10.0 μM imipramine | |
| K_{d} | 1.7 | nM |

TABLE 17
SERT binding affinity.

| Compound | SERT Kᵢ (μM) |
|---|---|
| 2S | 12 |
| 2R | 6.2 |
| 2-F-DCK | 40 |

### Example 19. Forced Swim Test in Rats

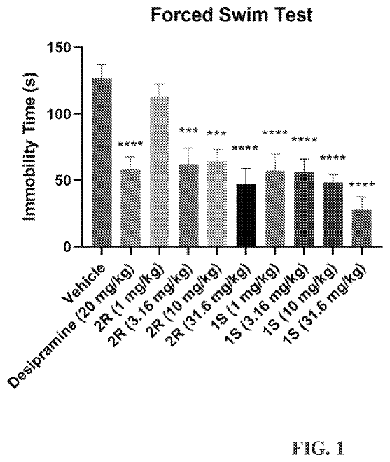

Disclosed compounds were tested in the forced swim test (FST) in rats with a 23.5-h pre-treatment time according to the following procedures. The compounds 2R and 1S reduced immobility time relative to vehicle control, indicative of an antidepressant-like effect (FIG. 1).

Animals. Male Sprague-Dawley rats, aged 8-10 weeks, were used in the experiments. Animals were housed in groups of 2 under controlled temperature (22±3° C.) and relative humidity (30-70%) conditions, with 12-hour light/dark cycles, and with ad libitum food and water. All efforts were made to minimize suffering.

Drugs and Drug Administration. Test compounds, saline vehicle, and the positive control desipramine were administered subcutaneously (s.c.), with doses calculated based on the freebase. Normal saline was used as the vehicle for compounds provided as the HCl salt, while saline acidified with 1-2 molar equivalents of HCl was used as the vehicle for compounds provided as the freebase (to form the soluble HCl salt in situ). All compounds were administered at a volume of 5 mL/kg. Test compounds and vehicle were administered 0.5 h after the start of the training swim (Swim 1) and 23.5 h before the test swim (Swim 2). Desipramine was administered 3 times, at 23.5 h, 5 h, and 1 h before the test swim (Swim 2), each time at a dose of 20 mg/kg.

Forced Swim Test (FST). Animals were randomized based on body weight to ensure that inter-group variations were minimal and did not exceed ±20% of the mean body weight across the groups. Group size was n=10 per treatment. Rats were handled for about 2 min daily for the 5 days prior to the beginning of the experimental procedure. On the first day of the experiment (i.e. Day 0), post randomization, training swim sessions (Swim 1) were conducted between 12:00 and 18:00 h with all animals by placing rats in individual glass cylinders (46 cm tall×20 cm in diameter) containing 23-25° C. water 30 cm deep for 15 minutes. At the conclusion of Swim 1, animals were dried with paper towels, placed in heated drying cages for 15 minutes, and then returned to their home cages. Animals were then administered the appropriate drug or vehicle treatment(s), as described above. For clarity, a compound administration time of 23.5 h before Swim 2 means 0.5 h after the start of Swim 1 and 0.25 h after the completion of Swim 1 (i.e., immediately after return to the home cage). On Day 1 (i.e., 24 h after start of Swim 1), animals performed the test swim (Swim 2) for a period of 5 min but otherwise under the same conditions as Swim 1. During all swim sessions, the water was changed between each animal.

Behavioral scoring was conducted by observers who were blind to the treatment groups. Animals were continuously observed during Swim 2 and the total time spent engaging in the following behaviors was recorded: immobile, swimming, and climbing. A rat was judged to be immobile when it remained floating in the water without struggling and made only those movements necessary to keep its head above water. A rat was judged to be swimming when it made active swimming motions, more than necessary to merely maintain its head above water (e.g. moving around in the cylinder). A rat was judged to be climbing when it made active movements with its forepaws in and out of the water, usually directed against the walls.

Statistical Analysis. Data points are presented as the mean±standard error of the mean (SEM), Analysis was performed using GraphPad Prism 6. Comparisons between groups were performed using the one-way analysis of variance (ANOVA), followed by Dunnett's test for comparisons to vehicle.

### Example 20. Comparative Metabolism of Compound 2R and its Deuterated Counterpart 7R

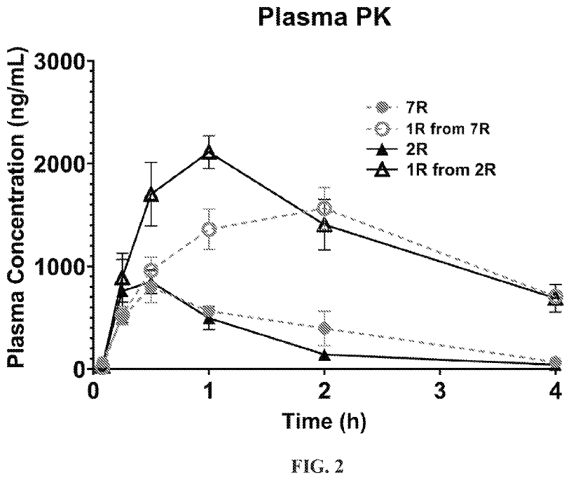

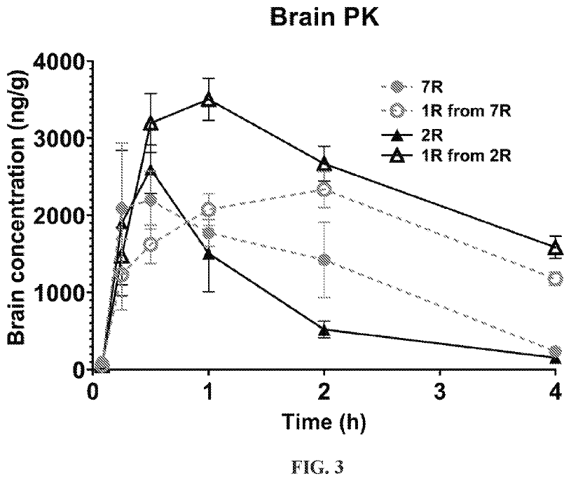

After oral administration in rats, deuterated compound 7R demonstrated greater exposure as quantified by area under the curve (AUC) in plasma and brain compared to its non-deuterated counterpart 2R (Table 18). This effect was most pronounced in the brain. Further, in terms of Cₘₐₓ, formation of the active metabolite 1R from 7R was attenuated compared to its formation from 2R, in both plasma and brain (Tables 18). This effect was most notable at earlier time points (e.g., 1 h or less), where levels of 1R derived from 2R were approximately 2-fold higher than levels of 1R derived from 7R (Tables 19 and 20, FIG. 2 and FIG. 3).

Animals. Male Sprague Dawley rats were used in these studies. Animals were randomly assigned to treatment groups and were fasted for 4 h before dosing.

Drugs. Test compounds 2R and 7R were dissolved in a vehicle consisting of normal saline. They were then administered orally (po) at a dose of 10 mg/kg (calculated based on freebase), and at a volume of 5 mL/kg body weight.

Sample Collection and Bioanalysis. Blood samples (approximately 60 μL) were collected under light isoflurane anesthesia (Surgivet®) from the retro orbital plexus at 0.08, 0.25, 0.5, 1, 2, 4, 8, and 24 h (4 animals per time point). Immediately after blood collection, plasma was harvested by centrifugation at 4,000 rpm for 10 min at 4° C. and samples were stored at −70±10° C. until bioanalysis. Following blood collection, animals were immediately sacrificed, the abdominal vena-cava was cut open, and the whole body was perfused from the heart using 10 mL of normal saline, and brain samples were collected from all animals. After isolation, brain samples were rinsed three times in ice-cold normal saline (for 5-10 seconds/rinse using ˜5-10 mL normal saline in disposable petri dish for each rinse) and dried on blotting paper. Brain samples were homogenized using ice-cold phosphate-buffered saline (pH 7.4) Total homogenate volume was three times the tissue weight. All homogenates were stored at −70±10° C. until bioanalysis. For bioanalysis, 25 μL aliquots of plasma/brain study samples or spiked plasma/brain calibration standards were added to individual pre-labeled micro-centrifuge tubes followed by 100 μL of an internal standard solution (glipizide, 500 ng/mL in acetonitrile) except for blanks, where 100 μL of acetonitrile was added. Samples were vortexed for 5 minutes and then centrifuged for 10 minutes at 4,000 rpm at 4° C. Following centrifugation, 100 μL of each clear supernatant was transferred to a 96 well plate and analyzed with a fit-for-purpose LC-MS/MS method, with authentic samples of each analyte used for calibration and identification. Concentrations of parent compound (2R or 7R) and metabolite 1R were determined in all samples.

Data Analysis. Pharmacokinetic parameters were estimated using the non-compartmental analysis tool of Phoenix® WinNonlin software (Ver 8.0).

TABLE 18
Pharmacokinetic parameters of 2R, 7R, and their metabolite 1R, in rat plasma and brain after oral administration of 2R and 7R.

| | | | Cₘₐₓ | Cₘₐₓ | AUC₀₋ₗₐₛₜ | AUC₀₋ₗₐₛₜ | t_{1/2} | t_{1/2} |
|---|---|---|---|---|---|---|---|---|
| Compound | | Dose | (plasma) | (brain) | (plasma) | (brain) | (plasma) | (brain) |
| Number | Structure | (mg/kg) | (ng/mL) | (ng/g) | (ng*min/mL) | (ng*min/g) | (min) | (min) |
| 7R | 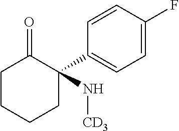 [SMILES: [2H]C([2H])([2H])N[C@@]1(c2ccc(F)cc2)CCCCC1=O] | 10 | 799 | 2197 | 99043 | 330347 | 65 | 67 |
| 1R as metabolite of 7R | 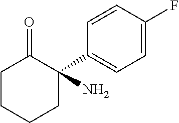 [SMILES: N[C@@]1(c2ccc(F)cc2)CCCCC1=O] | N/A | 1564 | 2335 | 503548 | 912802 | 182 | 185 |
| 2R | 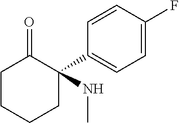 [SMILES: CN[C@@]1(c2ccc(F)cc2)CCCCC1=O] | 10 | 850 | 2599 | 72044 | 227268 | 70 | 71 |
| 1R as metabolite of 2R | 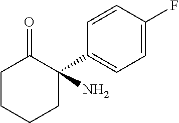 [SMILES: N[C@@]1(c2ccc(F)cc2)CCCCC1=O] | N/A | 2111 | 3502 | 517693 | 820350 | 182 | 121 |

TABLE 19
Mean plasma (ng/ml) concentrations of 7R, 2R, and metabolite 1R after oral administration of 2R and 7R (10 mg/kg).

| Compound | | | | | | | | |
|---|---|---|---|---|---|---|---|---|
| Nomber | Structure | Matrix | 0.083 h | 0.25 h | 0.5 h | 1 h | 2 h | 4 h |
| 7R | 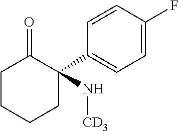 [SMILES: [2H]C([2H])([2H])N[C@@]1(c2ccc(F)cc2)CCCCC1=O] | plasma | 65 | 502 | 799 | 564 | 396 | 68 |
| 1R as metabolite of 7R | 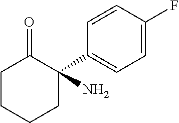 [SMILES: N[C@@]1(c2ccc(F)cc2)CCCCC1=O] | plasma | 16 | 525 | 960 | 1360 | 1564 | 705 |
| 2R | 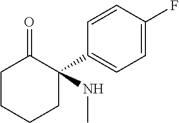 [SMILES: CN[C@@]1(c2ccc(F)cc2)CCCCC1=O] | plasma | 37 | 760 | 850 | 497 | 142 | 42 |
| 1R as metabolite of 2R | 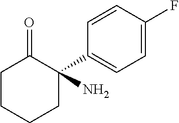 [SMILES: N[C@@]1(c2ccc(F)cc2)CCCCC1=O] | plasma | 31 | 889 | 1704 | 2111 | 1406 | 690 |

TABLE 20
Mean brain (ng/g) concentrations of 7R, 2R, and metabolite 1R after oral administration of 2R and 7R (10 mg/kg).

| Compound | | | | | | | | |
|---|---|---|---|---|---|---|---|---|
| Number | Structure | Matrix | 0.083 h | 0.25 h | 0.5 h | 1 h | 2 h | 4 h |
| 7R | 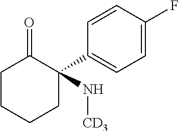 [SMILES: [2H]C([2H])([2H])N[C@@]1(c2ccc(F)cc2)CCCCC1=O] | brain | 98 | 2089 | 2197 | 1771 | 1423 | 236 |
| 1R as metabolite of 7R | 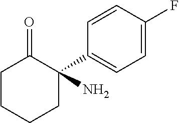 [SMILES: N[C@@]1(c2ccc(F)cc2)CCCCC1=O] | brain | 59 | 1233 | 1623 | 2075 | 2335 | 1176 |
| 2S | 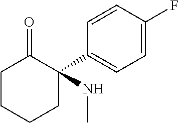 [SMILES: CN[C@@]1(c2ccc(F)cc2)CCCCC1=O] | brain | 43 | 1899 | 2599 | 1508 | 521 | 155 |
| 1R as metabolite of 2R | 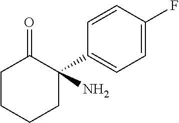 [SMILES: N[C@@]1(c2ccc(F)cc2)CCCCC1=O] | brain | 56 | 1473 | 3196 | 3502 | 2669 | 1608 |

### Example 21. Stability in Liver Microsomes

Compounds 2R, 2S, and 2-(2-fluorophenyl)-2-(methylamino)cyclohexan-1-one (2-F-DCK) were tested for stability in liver microsome preparations of various species (Table 21). Both 2R and 2S were much more stable (as indicated by lower intrinsic clearance, Clᵢₙₜ) than 2-F-DCK across multiple species, suggesting that 2R and 2S are likely to exhibit higher oral bioavailability than 2-F-DCK.

General Procedure. Briefly, test compounds (final concentration 1 μM) were incubated in duplicate with liver microsomes from male animals of the indicated species (final protein concentration 0.5 mg/mL) in 50 mM sodium phosphate buffer (pH 7.4) with or without NADPH (1 mM). Total incubation volume was 500 μL. At 0, 5, 15, 30, and 60 min, aliquots of 50 μL were withdrawn, quenched with acetonitrile (150 μL), and analyzed for parent compound remaining using a fit-for-purpose LC-MS/MS method. Intrinsic clearance and half-life were calculated. Clearance values below zero were rounded to zero.

TABLE 21
Microsomal stability of test compounds.

| | Clᵢₙₜ (μL/min/mg protein) | | | | |
|---|---|---|---|---|---|
| | CD-1 | SD | Beagle | Gottingen | Cynomolgus |
| Compound | Mouse | Rat | Dog | Minipig | Monkey |
| 2S | 3.50 | 14.1 | 7.30 | 20.3 | 15.3 |
| 2R | 0 | 16.2 | 10.5 | 16.4 | 9.50 |
| 2F-DCK | 10.9 | 111 | 30.0 | 56.2 | 212 |

### Example 22. Unblocking Kinetics at the NMDA Receptor

Compounds 2R, 2S, and 2-(2-fluorophenyl)-2-(methylamino)cyclohexan-1-one 2-F-DCK were tested for unblocking kinetics at the NMDA receptor in Xenopus laevis oocytes expressing recombinant human NMDA receptor (GRIN1/GRIN2B) (Table 22). Both 2R and 2S exhibited much shorter half-life for unblocking compared to 2-F-DCK. Since rapid dissociation kinetics from the NMDA receptor are believed to correlate with greater tolerability among NMDA receptor antagonists, these findings suggest that 2R and 2S are likely to be better tolerated than 2-F-DCK in terms of dissociative side effects.

Experimental Procedure. Oocytes were harvested from adult Xenopus laevis and incubated in 96 well plates for two to four days prior to recordings. Plasmids containing cDNA encoding for human NMDA receptor subunits GRIN1 and GRIN2B were transcribed using the mMessage mMachine T7 transcription kit (Ambion, USA). The Roboocyte automated injection system was used for injection of cRNA coding for hNMDA receptor subunits at a concentration of 100 ng/μL per subunit. Oocytes were clamped to a holding potential of −70 mV and induced currents after compound application were sampled at 200 Hz at room temperature. Agonist induced currents were recorded with a two-electrode voltage clamp. To determine the unblocking kinetics, glutamate and glycine (3 and 10 μM, respectively) were applied to the oocytes and the current recorded for 90 s. Then, compounds were applied at 3×IC50 for 120 s and the currents recorded.

TABLE 22
Unblocking half-lives of test compounds
at the NMDA receptor (n ≥ 5).

| Compound | Unblocking T_{1/2} ± SEM (s) |
|---|---|
| 2S | 27.0 ± 6.1 |
| 2R | 22.7 ± 2.3 |
| 2F-DCK | 86.3 ± 12.6 |

### Example 23. Comparative Pharmacokinetics of 2R, 2S, and 2-F-DCK in Mice

After oral administration in mice, the tested compounds demonstrated similar plasma pharmacokinetics (Table 23). However, in brain (Table 24), Compounds 2R and 2S exhibited substantially longer half-life (t_{1/2}), higher maximal concentrations (Cₘₐₓ), and greater total exposure as quantified by area under the curve (AUC) compared to 2-F-DCK Further, there was a substantial difference between the enantiomers of 2 in brain pharmacokinetics, with 2R exhibiting substantially longer half-life (t_{1/2}) and greater AUC compared to its enantiomer 2S.

Animals. Male C57BL/6 mice were used in these studies. Aminals were randomly assigned to treatment groups and were fasted for 4 h before dosing.

Drugs. Test compounds were dissolved in a vehicle consisting of normal saline, They were then administered orally (po) at a dose of 10 mg/kg (calculated based on freebase) and at a volume of 10 mL/kg body weight.

Sample Collection and Bioanalysis. Blood samples (approximately 60 μL) were collected under light isoflurane anesthesia (Surgivet®) from the retro orbital plexus at 0.08, 0.25, 0.5, 1, 2, 4, 8, and 24 h (4 animals per time point). Immediately after blood collection, plasma was harvested by centrifugation at 4,000 rpm for 10 min at 4° C. and samples were stored at −70±10° C. until bioanalysis. Following blood collection, animals were immediately sacrificed, the abdominal vena-cava was cut open, and the whole body was perfused from the heart using 10 mL of normal saline, and brain samples were collected from all animals. After isolation, brain samples were rinsed three times in ice-cold normal saline (for 5-10 seconds/rinse using ˜5-10 mL normal saline in disposable petri dish for each rinse) and dried on blotting paper. Brain samples were homogenized using ice-cold phosphate-buffered saline (pH 7.4). Total homogenate volume was three times the tissue weight. All homogenates were stored at −70±10° C. until bioanalysis. For bioanalysis, 25 μL aliquots of plasma/brain study samples or spiked plasma/brain calibration standards were added to individual pre-labeled micro-centrifuge tubes followed by 100 μL of an internal standard solution (glipizide, 500 ng/mL in acetonitrile) except for blanks, where 100 μL of acetonitrile was added. Samples were vortexed for 5 minutes and then centrifuged for 10 minutes at 4,000 rpm at 4° C. Following centrifugation, 100 μL of each clear supernatant was transferred to a 96 well plate and analyzed with a fit-for-purpose LC-MS/MS method, with authentic samples of each analyte used for calibration and identification.

Data Analysis. Pharmacokinetic parameters were estimated using the non-compartmental analysis tool of Phoenix® WinNonlin software (Ver 8.0).

TABLE 23
Selected oral pharmacokinetic parameters of disclosed compounds in plasma of mice.

| | | | Cₘₐₓ | | t_{1/2} |
|---|---|---|---|---|---|
| Compound | | Dose | (po) | AUC₀₋ₗₐₛₜ (po) | (po) |
| Number | Structure | (mg/kg) | (ng/ml) | (ng*min/mL)* | (min) |
| 2S | 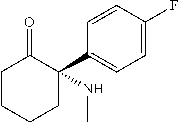 [SMILES: CN[C@]1(c2ccc(F)cc2)CCCCC1=O] | 10 | 1393 | 56382 | 89 |
| 2R | 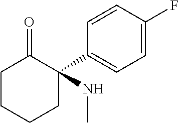 [SMILES: CN[C@@]1(c2ccc(F)cc2)CCCCC1=O] | 10 | 1182 | 67387 | 83 |
| 2-F-DCK | 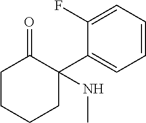 [SMILES: CNC1(c2ccccc2F)CCCCC1=O] | 10 | 1959 | 69032 | 69 |

TABLE 24
Selected oral pharmacokinetic parameters of disclosed compounds in brains of mice.

| | | | Cₘₐₓ | AUC₀₋ₗₐₛₜ | t_{1/2} |
|---|---|---|---|---|---|
| Compound | | Dose | (po) | (po) | (po) |
| Number | Structure | (mg/kg) | (ng/g) | (ng*min/g)* | (min) |
| 2S | 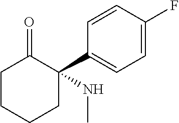 [SMILES: CN[C@]1(c2ccc(F)cc2)CCCCC1=O] | 10 | 3544 | 123388 | 131 |
| 2R | 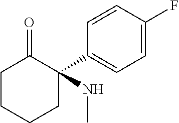 [SMILES: CN[C@@]1(c2ccc(F)cc2)CCCCC1=O] | 10 | 3277 | 200464 | 178 |
| 2-F-DCK | 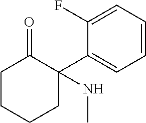 [SMILES: CNC1(c2ccccc2F)CCCCC1=O] | 10 | 1674 | 59119 | 41 |

While certain features of the present disclosure have been illustrated and described herein, many modifications, substitutions, changes, and equivalents will now occur to those of ordinary skill in the art. It is, therefore, to be understood that the appended claims are intended to cover all such modifications and changes as fall within the true spirit of the present disclosure.

What is claimed is:

1. An isolated, substantially enantiomerically pure compound selected from the group consisting of: 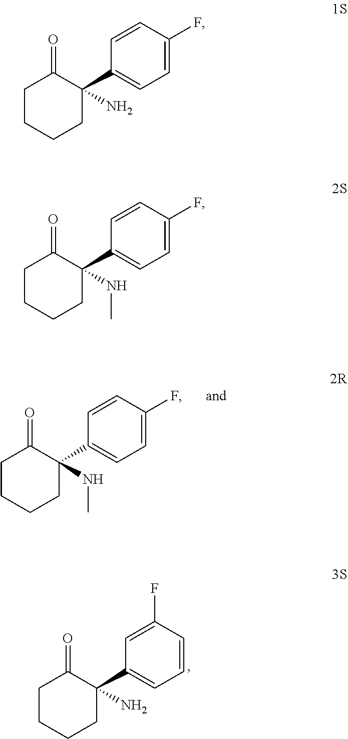 [SMILES: N[C@]1(c2ccc(F)cc2)CCCCC1=O ; CN[C@]1(c2ccc(F)cc2)CCCCC1=O ; CN[C@@]1(c2ccc(F)cc2)CCCCC1=O ; N[C@]1(c2cccc(F)c2)CCCCC1=O]
or a pharmaceutically acceptable salt thereof.

2. An enantiomeric compound selected from the group consisting of: 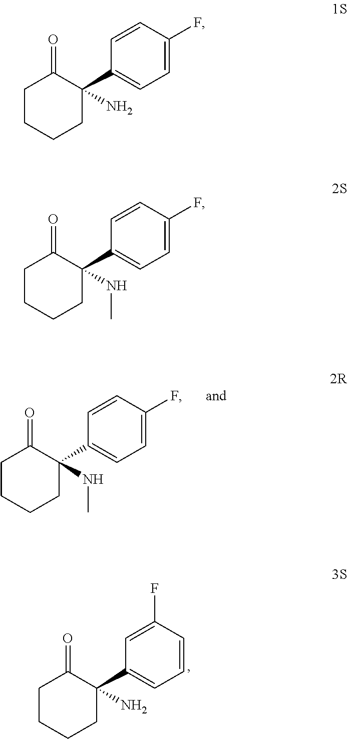 [SMILES: N[C@]1(c2ccc(F)cc2)CCCCC1=O ; CN[C@]1(c2ccc(F)cc2)CCCCC1=O ; CN[C@@]1(c2ccc(F)cc2)CCCCC1=O ; N[C@]1(c2cccc(F)c2)CCCCC1=O]
or a pharmaceutically acceptable salt thereof, wherein the enantiomeric compound is present in an enantiomeric mixture having at least 90%, at least 95%, or at least 99% of the enantiomeric compound.

3. The compound of claim 1 or 2, wherein the compound is: 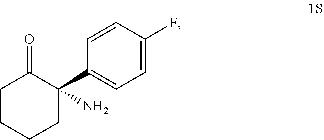 [SMILES: N[C@]1(c2ccc(F)cc2)CCCCC1=O]
or a pharmaceutically acceptable salt thereof.

4. The compound of claim 1 or 2, wherein the compound is: 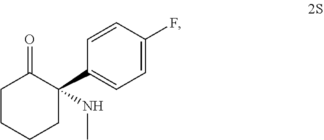 [SMILES: CN[C@]1(c2ccc(F)cc2)CCCCC1=O]
or a pharmaceutically acceptable salt thereof.

5. The compound of claim 1 or 2, wherein the compound is: 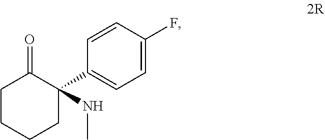 [SMILES: CN[C@@]1(c2ccc(F)cc2)CCCCC1=O]
or a pharmaceutically acceptable salt thereof.

6. The compound of claim 1 or 2, wherein the compound is: 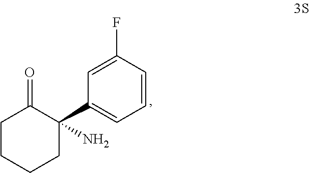 [SMILES: N[C@]1(c2cccc(F)c2)CCCCC1=O]
or a pharmaceutically acceptable salt thereof.

7. A pharmaceutical composition comprising a compound of any one of claims 1-6 and a pharmaceutically acceptable excipient.

8. The pharmaceutical composition of claim 7, wherein the composition is an oral composition.

9. A composition comprising an enantiomeric mixture of a compound selected from the group consisting of: 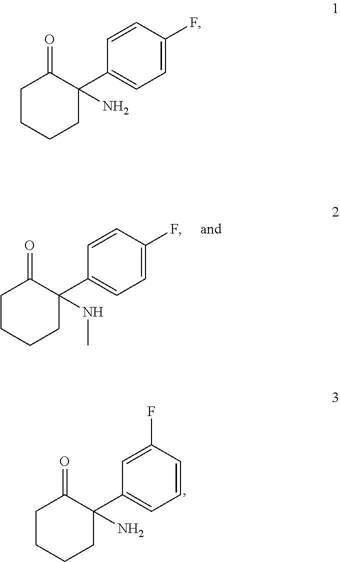 [SMILES: NC1(c2ccc(F)cc2)CCCCC1=O ; CNC1(c2ccc(F)cc2)CCCCC1=O ; NC1(c2cccc(F)c2)CCCCC1=O]
or a pharmaceutically acceptable salt thereof, wherein the enantiomeric mixture has a significantly greater amount of the enantiomer having the higher binding affinity at the NMDA receptor MK-801 site.

10. A composition comprising an enantiomeric mixture of the compound: 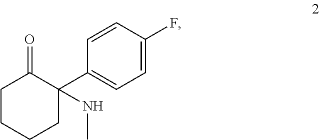 [SMILES: CNC1(c2ccc(F)cc2)CCCCC1=O]
or a pharmaceutically acceptable salt thereof, wherein the enantiomeric mixture has a significantly greater amount of the enantiomer having the lower binding affinity at the NMDA receptor MK-801 site.

11. A method of treating depression, anxious depression, a mood disorder, an anxiety disorder, or a substance use disorder and any symptom or disorders associated therewith in a subject in need thereof, the method comprising administering to the subject in need thereof an effective amount of a compound or composition of any one of claims 1-10.

12. The method of claim 11, wherein the compound or composition is orally administered.

13. A method of treating depression or anxious depression in a subject in need thereof, the method comprising administering to the subject in need thereof an effective amount of a compound or composition of any one of claims 1-10.

14. The method of claim 13, wherein the compound or composition is orally administered.

15. A method of treating depression, anxious depression, a mood disorder, an anxiety disorder, or a substance use disorder and any symptom or disorders associated therewith in a subject in need thereof, the method comprising administering to the subject in need thereof an effective amount of a compound selected from the group consisting of: 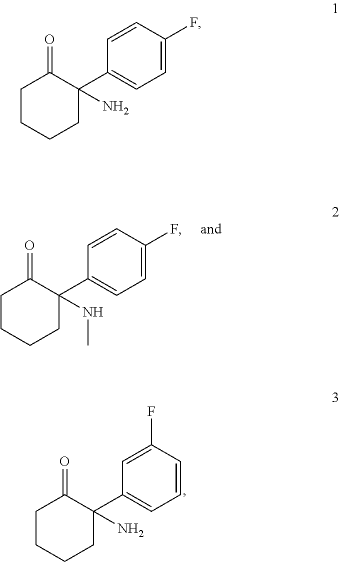 [SMILES: NC1(c2ccc(F)cc2)CCCCC1=O ; CNC1(c2ccc(F)cc2)CCCCC1=O ; NC1(c2cccc(F)c2)CCCCC1=O]
or a pharmaceutically acceptable salt thereof.

16. The method of claim 15, wherein the compound or composition is orally administered.
